(19) United States
(12) Patent Application Publication
(10) Pub. No.: US 2025/0144054 A1
(43) Pub. Date: May 8, 2025

(54) SMALL MOLECULE STAT3 INHIBITOR FOR TREATING TRIPLE NEGATIVE BREAST CANCER

(71) Applicants: UNIVERSITY OF MARYLAND, BALTIMORE, Baltimore, MD (US); UNIVERSITY OF FLORIDA RESEARCH FOUNDATION, INC., Gainsville, FL (US)

(72) Inventors: Jiayuh LIN, Ellicott City, MD (US); Li PAN, Baltimore, MD (US); Chenglong LI, Gainesville, FL (US)

(73) Assignees: UNIVERSITY OF MARYLAND, BALTIMORE, Baltimore, MD (US); UNIVERSITY OF FLORIDA RESEARCH FOUNDATION, INC., Gainsville, FL (US)

(21) Appl. No.: 18/837,949

(22) PCT Filed: Feb. 15, 2023
(86) PCT No.: PCT/US2023/062633

§ 371 Date: Aug. 13, 2024

Related U.S. Application Data
(60) Provisional application No. 63/310,229, filed on Feb. 15, 2022.

(51) Int. Cl. A61K 31/18; A61K 45/06; A61P 35/00

(52) U.S. Cl. CPC A61K 31/18; A61K 45/06; A61P 35/00

(57) ABSTRACT

Methods of using the small molecule STAT3 inhibitor LLL 12B in the treatment of cancer, such as triple-negative breast cancer (TNBC) and pancreatic cancer, either alone or in combination with additional therapeutic agents, such as PARP inhibitors and CDK inhibitors, are provided.

### BACKGROUND OF INVENTION

Breast cancer is a heterogeneous disease and has become the most diagnosed cancer in women worldwide [1]. It is composed of different subtypes [2], one such subtype being triple-negative breast cancer (TNBC), where tumors lack the expression of estrogen receptor-alpha (ERα), progesterone receptor (PR), and HER2 [3]. TNBC accounts for approximately 15%-20% of all breast cancers, and unlike other subtypes of breast cancer, TNBC is more aggressive, has a greater distant metastasis potential, and a poorer survival rate [4].

Due to genomic heterogeneity and lack of validated biomarkers, TNBC treatment is very challenging. The main treatment for early-stage TNBC is chemotherapy followed by surgery. Recently, poly-ADP-ribose polymerase (PARP) inhibitors were approved for the treatment of metastatic breast cancers with germline BRCA mutations [5]. Immune checkpoint inhibitors (ICIs) targeting programmed cell death protein 1 (PD-1)/programmed cell death-ligand 1 (PD-L1), such as pembrolizumab, in combination with chemotherapy, were also approved for patients with metastatic TNBC expressing PD-L1 [6,7]. Despite the encouraging results of PARP inhibitors and immunotherapy for TNBC, their use remains limited for patients with germline BRCA mutations and PD-L1 expression. Therefore, developing novel therapeutic targets is crucial for TNBC treatment.

Recent advances have been made in understanding the molecular heterogeneity of TNBC and several potential therapeutic targets such as PI3K/AKT/mTOR (phosphatidylinositol 3-kinase/AKT/mammalian target of the rapamycin), CDKs (cyclin-dependent Kinases), EGFR (epidermal growth factor receptor), VEGFR (vascular endothelial growth factor receptor), AR (androgen receptor), and STAT3 (signal transducer and activator of transcription 3) have been explored [8-11]. Notably, STAT3 is persistently activated in various cancers, including breast cancer, and is often activated and required for the growth and survival of TNBC cells [12-14].

Accumulating evidence shows that persistent STAT3 signaling is crucial for cell proliferation [15], cell invasion and migration [16], chemoresistance [17], angiogenesis, immune regulation [18], cell metabolism [19], and the maintenance of stem cell properties in TNBC [11,14]. In addition, TNBC patients with elevated expression of p-STAT3 have been associated with a decreased probability of relapse-free survival compared to those with low expression of p-STAT3 [20,21]. Therefore, targeting STAT3 is likely to be a potential strategy for TNBC therapy. A variety of STAT3 inhibitors with different mechanisms have been developed and a few of them are in clinical trials, but there are no FDA-approved STAT3 inhibitors for treatment to date [22]. The development of specific and effective novel STAT3 inhibitors for potential cancer prevention and therapy is desirable.

### BRIEF SUMMARY OF INVENTION

The present invention relates to a small molecule STAT3 inhibitor that may be used, inter alia, for the treatment of cancer, such as triple-negative breast cancer (TNBC), either alone or in combination with additional therapeutic agents, such as PARP inhibitors and CDK inhibitors. The STAT3 inhibitor is termed “LLL12B” herein and the structure of the compound is as follows.

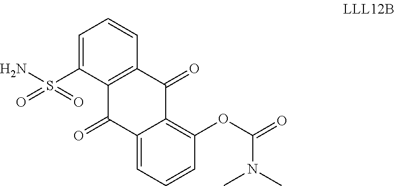 [SMILES: CN(C)C(=O)Oc1cccc2c1C(=O)c1cccc(S(N)(=O)=O)c1C2=O]

A first embodiment of the invention provides a method of treating cancer in a subject, comprising administering a therapeutically-effective amount of LLL12B to a subject in need thereof, thereby treating cancer in a subject.

Examples of cancers that may be treated include, but are not limited to, cancers having cells that persistently or constitutively expresses STAT3 (signal transducer and activator of transcription 3). Examples of such cancers include, for example, breast cancer, such as triple-negative breast cancer (TNBC), and pancreatic cancer.

In one aspect of this embodiment, the invention provides a method of treating TNBC in a subject, comprising administering a therapeutically-effective amount of LLL12B to a subject having TNBC, thereby treating TNBC in a subject.

In another aspect of this embodiment, the invention provides a method of treating pancreatic cancer in a subject, comprising administering a therapeutically-effective amount of LLL12B to a subject having pancreatic cancer, thereby treating pancreatic cancer in a subject.

In some aspect of this embodiment, the therapeutically-effective amount of LLL12B is sufficient to achieve one or more of (i) induce apoptosis in cells of the cancer, (ii) inhibit or block growth of cells of the cancer, (iii) inhibit or block migration of cells of the cancer, (iv) inhibit or block STAT3 activity in cells of the cancer, (v) inhibit or block STAT3 signaling in cells of the cancer, (vi) inhibit or block STAT3 phosphorylation in cells of the cancer, and (vii) inhibit or block STAT3 phosphorylation-induction activation in cells of the cancer.

In other aspects of this embodiment, the therapeutically-effective amount of LLL12B is an amount ranging from about 1 mg/kg to 20 mg/kg, body weight.

A second embodiment of the invention provides a method of treating cancer in a subject, comprising administering a therapeutically-effective amount of LLL12B and one or more additional therapeutic agents to a subject in need thereof, thereby treating cancer in a subject.

Examples of additional therapeutic agents include, but are not limited to, PARP inhibitors and CDK inhibitors. Suitable PARP inhibitors include, for example, talazoparib, olaparib, rucaparib, veliparib and niraparib. Suitable CDK inhibitors include, for example, the CDK4/6 inhibitor abemaciclib, palbociclib, ribociclib, Alvocidib, Dinaciclib, P276-00, AT7519 and Roniciclib.

Examples of cancers that may be treated include, but are not limited to, cancers having cells that persistently or constitutively expresses STAT3 (signal transducer and activator of transcription 3). Examples of such cancers include, for example, breast cancer, such as triple-negative breast cancer (TNBC), and pancreatic cancer.

In one aspect of this embodiment, the invention provides a method of treating TNBC in a subject, comprising administering a therapeutically-effective amount of LLL12B and one or more PARP inhibitors to a subject having TNBC, thereby treating TNBC in a subject.

In another aspect of this embodiment, the invention provides a method of treating pancreatic cancer in a subject, comprising administering a therapeutically-effective amount of LLL12B and one or more PARP inhibitors to a subject having pancreatic cancer, thereby treating pancreatic cancer in a subject.

In a further aspect of this embodiment, the invention provides a method of treating TNBC in a subject, comprising administering a therapeutically-effective amount of LLL12B and one or more CDK inhibitors to a subject having TNBC, thereby treating TNBC in a subject.

In yet another aspect of this embodiment, the invention provides a method of treating pancreatic cancer in a subject, comprising administering a therapeutically-effective amount of LLL12B and one or more CDK inhibitors to a subject having pancreatic cancer, thereby treating pancreatic cancer in a subject.

LLL12B and the additional therapeutic agent(s) may be administered in any order, alone or in combination, sequentially or concurrently, with overlapping or non-overlapping periods of administration.

In some aspects of this embodiment, the combination of LLL12B and the additional therapeutic agent has a synergistic therapeutic effect on the cancer. Thus, the therapeutic effect of the combination of LLL12B and the additional therapeutic agent may be greater than the additive effects seen when either LLL12B or the additional therapeutic agent is administered alone.

In some of this embodiment, the therapeutically-effective amount of LLL12B is sufficient to achieve one or more of (i) induce apoptosis in cells of the cancer, (ii) inhibit or block growth of cells of the cancer, (iii) inhibit or block migration of cells of the cancer, (iv) inhibit or block STAT3 activity in cells of the cancer, (v) inhibit or block STAT3 signaling in cells of the cancer, (vi) inhibit or block STAT3 phosphorylation in cells of the cancer, and (vii) inhibit or block STAT3 phosphorylation-induction activation in cell of the cancer.

In other aspects of this embodiment, the therapeutically-effective amount of LLL12B is an amount ranging from about 1 mg/kg to 20 mg/kg, body weight.

In aspects of this embodiment, the amount of the PARP inhibitor is an amount ranging from about 0.05 to about 15 mg/kg, body weight.

In aspects of this embodiment, the amount of the CDK inhibitor is an amount ranging from about 10 to about 250 mg/kg, body weight.

A third embodiment of the invention is directed to related methods of inhibiting STAT3 activity, either in vitro or in vivo. Thus, in one aspect, the invention provides a method of inhibiting STAT3 activity in vitro, comprising contacting cells expressing STAT3 with an amount of LLL12B effective to inhibit STAT3 activity, thereby inhibiting STAT3 activity in vitro.

In a related aspect, the invention provides a method of inhibiting STAT3 activity in vivo, comprising administering to a subject having cells expressing STAT3 an amount of LLL12B effective to inhibit STAT3 activity, thereby inhibiting STAT3 activity in vivo.

In each aspect of this embodiment, the cells expressing STAT3 may be cells of a cancer. Examples of such cancers include, but are not limited to, cancers that persistently or constitutively expresses STAT3 (signal transducer and activator of transcription 3). Examples of such cancers include, for example, breast cancer, such as triple-negative breast cancer (TNBC), and pancreatic cancer.

In each aspect of this embodiment, the effective amount of LLL12B for in vitro methods is an amount ranging from about 0.25 μM to about 5 μM, culture media.

In each aspect of this embodiment, the effective amount of LLL12B for in vivo methods is an amount ranging from about 1 mg/kg to 20 mg/kg, body weight.

In in vitro aspects of this embodiment, the cells may also be contacted with an effective amount of one or more additional therapeutic agents, such as a PARP inhibitor or a CDK inhibitor. The effective amount of PARP inhibitors for in vitro methods is an amount ranging from about 0.01 μM to about 20 μM, culture media. The effective amount of CDK inhibitors for in vitro methods is an amount ranging from about 0.5 μM to about 5 μM, culture media.

In in vivo aspects of this embodiment, an effective amount of one or more additional therapeutic agents may also be administered to the subject, such as a PARP inhibitor or a CDK inhibitor. The effective amount of PARP inhibitors for in vivo methods is an amount ranging from about 0.05 to about 15 mg/kg, body weight. The effective amount of CDK inhibitors for in vivo methods is an amount ranging from about 10 to about 250 mg/kg, body weight.

When included, the combination of LLL12B and the one or more additional therapeutic agent may have a synergistic effect on inhibiting STAT3 activity, whether in vitro or in vivo.

A fourth embodiment of the invention is directed to related methods of inhibiting growth of cells expressing STAT3, either in vitro or in vivo. Thus, in one aspect, the invention provides a method of inhibiting growth of cells expressing STAT3 in vitro, comprising contacting cells expressing STAT3 with an amount of LLL12B effective to inhibit growth of the cells, thereby inhibiting growth of cells expressing STAT3 in vitro.

In a related aspect, the invention provides a method of inhibiting growth of cells expressing STAT3 in vivo, comprising administering to a subject having cells expressing STAT3 an amount of LLL12B effective to inhibit growth of the cells, thereby inhibiting growth of cells expressing STAT3 in vivo.

In each aspect of this embodiment, the cells may be cancer cells. Examples of such cancers include, but are not limited to, cancers that persistently or constitutively expresses STAT3 (signal transducer and activator of transcription 3). Examples of such cancers include, for example, breast cancer, such as triple-negative breast cancer (TNBC), and pancreatic cancer.

In each aspect of this embodiment, the effective amount of LLL12B for in vitro methods is an amount ranging from about 0.25 μM to about 5 μM, culture media.

In each aspect of this embodiment, the effective amount of LLL12B for in vivo methods is an amount ranging from about 1 mg/kg to 20 mg/kg, body weight.

In in vitro aspects of this embodiment, the cells may also be contacted with an effective amount of one or more additional therapeutic agents, such as a PARP inhibitor or a CDK inhibitor. The effective amount of PARP inhibitors for in vitro methods is an amount ranging from about 0.01 μM to about 20 μM, culture media. The effective amount of CDK inhibitors for in vitro methods is an amount ranging from about 0.5 μM to about 5 μM, culture media.

In in vivo aspects of this embodiment, an effective amount of one or more additional therapeutic agents may also be administered to the subject, such as a PARP inhibitor or a CDK inhibitor. The effective amount of PARP inhibitors for in vivo methods is an amount ranging from about 0.05 to about 15 mg/kg, body weight. The effective amount of CDK inhibitors for in vivo methods is an amount ranging from about 10 to about 250 mg/kg, body weight.

When included, the combination of LLL12B and the one or more additional therapeutic agent may have a synergistic effect on inhibiting growth of cells expressing STAT3, whether in vitro or in vivo.

In each of the aspects and embodiments of the invention, the effective amount of LLL12B and the therapeutically-effective amount of LLL12B may be administered to a subject in the form of a pharmaceutical composition comprising LLL12B and one or more pharmaceutically acceptable diluents and/or excipients. The pharmaceutical composition comprising LLL12B may be formulated for oral delivery, although it should be understood that the invention is not limited to such formulations or related routes of delivery.

In each of the aspects and embodiments of the invention, the subject may be a human.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1 shows knockdown of STAT3 inhibited TNBC cell viability. MDA-MB-231, SUM159, and 4T1 cells were transfected with control siRNA and STAT3 siRNA for 72 hours and cell viability was evaluated by MTT assay (A). The expression levels of P-STAT3 (Y705), STAT3, and Cleaved Caspase-3 were analyzed by Western blot (B). GAPDH served as a protein loading control. *** P<0.001.

FIG. 2 shows LLL12B inhibited STAT3 nuclear translocation and IL-6 induced STAT3 phosphorylation. (A) The chemical structures of LLL12B and LLL12. The carbamate ester bond of LLL12B is hydrolytically cleaved by the tumor-associated plasmin to release LLL12. (B) SUM159 cells were seeded in a 6-well plate and treated with LLL12B for 8 hours. Cells were stained with Phospho-Stat3 (Tyr705) and DAPI (indicated by arrows). Green: P-STAT3 (Y705); Blue: DAPI. T47D (C) and MDA-MB-231 (D) breast cancer cells were pretreated with DMSO or LLL12B in a serum-free medium for 2 hours and stimulated with 50 ng/ml of IL-6, IFN-γ, or EGF for additional 30 minutes. Cells were collected and analyzed by Western blot. GAPDH served as a protein loading control.

FIG. 3 shows LLL12B inhibited STAT3 phosphorylation and induced apoptosis in TNBC cells. (A) MDA-MB-231, SUM159, and 4T1 cell lines were treated with DMSO or LLL12B overnight and the expression levels of P-STAT3 (Y705), STAT3, Cyclin D1, and Cleaved Caspase-3 were analyzed by Western blot. GAPDH served as a protein loading control. (B) MDA-MB-231 cells were treated with DMSO or C188-9 overnight and the expression levels of P-STAT3 (Y705), STAT3, Cyclin D1, and Cleaved Caspase-3 were analyzed by Western blot. (C) MDA-MB-231, SUM159, and 4T1 cells were seeded in a 96-well plate and treated with DMSO or LLL12B for 5 hours and a Caspase-3/7 Fluorescence Assay Kit was used to detect the activation of caspase-3/7. (D) MDA-MB-231, SUM159, and 4T1 cell lines were treated with DMSO or LLL12B for 8-12 hours, and flow cytometry was performed to analyze annexin V and PI staining. The quantification of apoptotic cells was shown in the right panel. ** P<0.01, *** P<0.001 and ****P<0.0001.

FIG. 4 shows LLL12B suppressed colony formation of TNBC cells. MDA-MB-231 (A), SUM159 (B), and 4T1 (C) cells were seeded in a 6-well plate and treated with DMSO or LLL12B for 24 hours and recovered in a drug-free culture medium for 7 days. Representative images of colony formation were shown in the left panel and the colony number was quantified and shown in the right panel. ** P<0.01, *** P<0.001 and **** P<0.0001.

FIG. 5 shows LLL12B inhibited migration in TNBC cells. (A) Wound healing assay was performed to detect the cell migration in MDA-MB-231, SUM159, and 4T1 cell lines with LLL12B treatment. Cell images showing the initial and final scratches. (B) Quantification analysis of the wound area was shown. * P<0.05, ** P<0.01, *** P<0.001 and **** P<0.0001.

FIG. 6 shows LLL12B suppressed MDA-MB-231 tumor growth in vivo. MDA-MB-231 cells were inoculated into the 4th mammary fat pads, and Vehicle or LLL12B (2.5 mg/kg) was orally administered every day for 28 days. Tumor volumes were measured every 2 days (A). (B) Body weights were measured every 2 days. (C) Tumors were removed after 28 days of treatment and tumor weights were measured. Tumor samples are shown in the bottom panel. (D) The expression levels of P-STAT3 (Y705), STAT3, and Cyclin D1 in the vehicle and LLL12B treated tumor samples were analyzed by Western blot. GAPDH served as a protein loading control. ** P<0.01, **** P<0.001.

FIG. 7 shows STAT3 inhibitor LLL12B inhibited cell viability of human pancreatic cancer cell lines HPAC (A) and PANC-1 (B). The human pancreatic cancer cells were treated with LLL12B for 3 days and the cell viability was determined by MTT assay.

FIG. 8 shows STAT3 inhibitor LLL12B inhibited cell migration of human pancreatic cancer cell lines HPAC. The human pancreatic cancer cells were treated with LLL12B for 17 hours and the cell migration was determined by wound healing assay, with percent migration shown graphically (A) and via microscopic studies (B).

FIG. 9 shows STAT3 inhibitor LLL12B in combination with PARP inhibitor Talazoparib synergistically inhibited cell viability in six human triple-negative breast cancer cells (A-F) (L: LLL12B; T: Talozoparib). Cells were treated with LLL12B and Talazoparib for 3 days and the cell viability was determined by MTT assay. (L0.1:0.1 μM LLL12B; L0.5:0.5 μM LLL12B; T0.5:0.5 μM Talozoparib; T0.01:0.01 μM Talozoparib; T2.5:2.5 μM Talozoparib; T20: 20 μM Talozoparib; T10: 10 μM Talozoparib).

FIG. 10 shows STAT3 inhibitor LLL12B in combination with PARP inhibitor Talazoparib synergistically inhibited cell growth in four human triple-negative breast cancer cells (A-D) (L: LLL12B; T: Talozoparib).

FIG. 11 shows STAT3 inhibitor LLL12B in combination with PARP inhibitor Talazoparib synergistically inhibited colony formation in human triple-negative breast cancer cells.

FIG. 12 shows STAT3 inhibitor LLL12B in combination with PARP inhibitor Talazoparib synergistically inhibited cell migration of human triple-negative breast cancer cell lines SUM149 and HCC1937 (A-B) (L: LLL12B; T: Talozoparib). Cell migration was determined by wound healing assay (L0.5:0.5 μM LLL12B; L0.25:0.25 μM LLL12B; T0.5:0.5 μM Talozoparib; T10: 10 μM Talozoparib).

FIG. 13 shows STAT3 inhibitor LLL12B in combination with PARP inhibitor Talazoparib synergistically inhibited cell migration of human triple-negative breast cancer cell lines MDA-MB-436 and BT-20 (A-B) (L: LLL12B; T: Talozoparib). Cell migration was determined by wound healing assay (L0.5:0.5 μM LLL12B; T10: 10 μM Talozoparib; T20: 20 μM Talozoparib).

FIG. 14 shows STAT3 inhibitor LLL12B in combination with PARP inhibitor Talazoparib synergistically inhibited cell migration of human triple-negative breast cancer cell lines MDA-MB-231 and SUM159 (A-B) (L: LLL12B; T: Talozoparib). Cell migration was determined by wound healing assay (L0.5:0.5 μM LLL12B; T10: 10 μM Talozoparib; T20: 20 μM Talozoparib).

FIG. 15 shows STAT3 inhibitor LLL12B in combination with PARP inhibitor Talazoparib synergistically induced cell apoptosis of human triple-negative breast cancer cell line SUM149 (A-B) (L: LLL12B; T: Talozoparib). Cell apoptosis was determined by Annexin V and PI staining using flow cytometry (L0.25:0.25 μM LLL12B; T0.025:0.025 μM Talozoparib).

FIG. 16 shows LLL12B sensitizes to Talazoparib in a SUM149 xenograft model (A-D) (V: Vehicle; L: LLL12B; T: Talazoparib).

FIG. 17 shows STAT3 inhibitor LLL12B in combination with CDK4/6 inhibitor Abemaciclib synergistically inhibited cell viability in human triple-negative breast cancer cells (A-D) (L: LLL12B; A: Abemaciclib).

FIG. 18 shows STAT3 inhibitor LLL12B in combination with CDK4/6 inhibitor Abemaciclib synergistically inhibited colony formation in human triple-negative breast cancer cells.

FIG. 19 shows STAT3 inhibitor LLL12B in combination with CDK4/6 inhibitor Abemaciclib synergistically inhibited cell migration in human triple-negative breast cancer cells (A-B) (L: LLL12B; A: Abemaciclib).

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

STAT3 is one of the STAT family proteins including STAT1, STAT2, STAT3, STAT4, STAT5A, STAT5B, and STAT6. They share a homologous domain structure composed of an amino-terminal domain, a coiled-coil domain, a DNA-binding domain, an a-helical linker domain, an SRC-homology 2 (SH2) domain, and a carboxy-terminal transactivation domain [33-35]. These similarities make it more challenging to target STAT3 specifically. In particular, both STAT1 and STAT3 are involved in tumorigenesis but play opposite roles [36]. The canonical STAT3 signaling pathway is activated by the binding of cytokines or growth factors to their corresponding receptors [37]. Among them, IL-6 is critical for STAT3 activation in human breast cancer [13,38].

STAT3 is a valuable target for anticancer drug development. Various strategies targeting STAT3 directly or upstream kinases indirectly have been tested, including peptides, small molecules, oligonucleotides, and natural compounds [22,39-41]. Because of the off-target toxicities of STAT3 upstream kinases inhibitors, lacking stability of peptides, and lacking effective delivery of oligonucleotides, small molecular inhibitors targeting STAT3 directly remain to be the most common approach. To date, the majority of small molecule STAT3 inhibitors are designed to target the SH2 domain which is essential for STAT3 dimerization. A number of small molecule inhibitors of STAT3 targeting the SH2 domain have been reported and shown anti-tumor activity, such as Stattic [42], S31-201 [43], C188-9 [32], and Bt354 [44]. Several STAT3 inhibitors targeting the SH2 domain have been developed using computer-aided drug design, including LLL12, LLY17, and LLL12B [23-25,27,28]. LLL12B is a prodrug of LLL12 with superior in vivo pharmacokinetic properties compared to LLL12 [25,28]. It has been shown that LLL12B induced apoptosis, suppressed tumor growth, and exhibited synergistic activity in combination with cisplatin or irradiation in medulloblastoma cells [25,31]. In addition, LLL12B sensitized ovarian cancer cells to paclitaxel and cisplatin [45].

With advances in computer-aided drug design, several selective STAT3 inhibitors were developed using the advanced multiple ligand simultaneous docking method (AMLSD) [23-25]. It was found that LLL12B, a prodrug of LLL12, is activated by the tumor-associated plasmin through the hydrolytic cleavage of the carbamate ester bond to release LLL12 [26]. LLL12B binds directly to the pTyr705 binding site of STAT3 with improved in vivo pharmacokinetic properties relative to the parent drug LLL12, which supports that it has superior and selective inhibition of STAT3 [25,27,28].

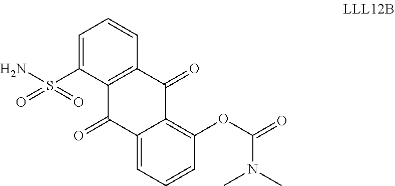 [SMILES: CN(C)C(=O)Oc1cccc2c1C(=O)c1cccc(S(N)(=O)=O)c1C2=O]

As disclosed herein, LLL12B was tested in TNBC cells and the results show that targeting STAT3 with LLL12B induced apoptosis, suppressed colony formation, migration, and tumor growth in TNBC cells. LLL12B also selectively inhibited IL-6 mediated STAT3 activation but had little effect on IFN-γ-mediated STAT1 activation and EGF-mediated ERK activation, supporting LLL12B as a selective STAT3 inhibitor.

Taken together, the findings demonstrate the orally bioavailable STAT3 inhibitor, LLL12B, as a novel therapeutic agent for cancer treatment, such as TNBC therapy.

### Treating Cancer

As summarized above, the present invention is directed to methods of treating cancer in a subject via the administration of LLL12B, either alone or in combination with additional therapeutic agents, such PARP (poly(ADP-ribose) polymerase) inhibitors and/or CDK (cyclin-dependent kinase) inhibitors. LLL12B has been found to induce apoptosis, suppress colony formation, migration, and tumor growth in cancer cells, such as those of TNBC and pancreatic cancer, for example.

Thus, the invention relates to methods of treating cancer in a subject. In one embodiment, the method comprises administering a therapeutically-effective amount of LLL12B to a subject in need thereof, i.e. a subject having cancer or suspect of having cancer, thereby treating cancer in a subject. In another embodiment, the invention comprises administering a therapeutically-effective amount of LLL12B and one or more additional therapeutic agents to a subject in need thereof, i.e. a subject having cancer or suspect of having cancer, thereby treating cancer in a subject.

In each of these and other embodiments of the invention, the cancers that may be treated are those characterized by persistent or constitutive expression (or over expression) of STAT3 (signal transducer and activator of transcription 3). STAT3, a member of the STAT protein family, mediates gene expression in response to cellular stimuli, playing a key role in cellular processes such as cell growth and apoptosis. Cytokines and growth factors induce STAT3 activation via phosphorylation at TYR705 or SER727. The activated protein forms homo- or heterodimers, and translocates to the cell nucleus where it acts as a transcription activator.

The cancers that may be treated by the methods of the invention include, but are not limited to, breast cancer, such as triple-negative breast cancer (TNBC) and pancreatic cancer.

The therapeutically-effective amount of LLL12B administered to a subject may be defined as an amount sufficient to achieve one or more of (i) induce apoptosis in cells of the cancer, (ii) inhibit or block growth of cells of the cancer, (iii) inhibit or block migration of cells of the cancer, (iv) inhibit or block STAT3 activity in cells of the cancer, (v) inhibit or block STAT3 signaling in cells of the cancer, (vi) inhibit or block STAT3 phosphorylation in cells of the cancer, and (vii) inhibit or block STAT3 phosphorylation-induction activation in cells of the cancer.

The therapeutically-effective amount of LLL12B administered to a subject may alternatively, or in addition, be defined as a discrete dose of the compound ranging from about 0.01 to about 100 mg/kg body weight of the subject. In some embodiments, the therapeutically-effective amount of LLL12B ranges from about 0.1 to about 50 mg/kg body weight, or about 1 to about 20 mg/kg body weight, or about 2 to about 10 mg/kg body weight. The specific amounts may vary depending on the cancer being treated.

PARP inhibitors that may be used in the methods are include, but are not limited to, talazoparib, olaparib, rucaparib, veliparib and niraparib.

CDK inhibitors that may be used in the methods are include, but are not limited to, abemaciclib, palbociclib, ribociclib, Alvocidib, Dinaciclib, P276-00, AT7519 and Roniciclib.

The effective amount of the PARP inhibitor that may be used in the methods of the invention generally ranging from about 0.01 to about 50 mg/kg body weight of the subject. In some embodiments, the amount of the PARP inhibitor ranges from about 0.01 to about 25 mg/kg body weight, or about 0.05 to about 15 mg/kg body weight, or about 0.1 to about 10 mg/kg body weight. The specific amounts may vary depending on the cancer being treated and the identity of the PARP inhibitor(s). The effective amount of the PARP may alternatively, or in addition, be defined as the amount required to augment the activity of LLL12B. The effective amount of the PARP may alternatively, or in addition, also be defined as the amount required to induce a synergistic effect with LLL12B.

The effective amount of the CDK inhibitor that may be used in the methods of the invention generally ranging from about 1 to about 1000 mg/kg body weight of the subject. In some embodiments, the amount of the CDK inhibitor ranges from about 5 to about 500 mg/kg body weight, or about 10 to about 250 mg/kg body weight, or about 50 to about 100 mg/kg body weight. The specific amounts may vary depending on the cancer being treated and the identity of the CDK inhibitor(s). The effective amount of the CDK may alternatively, or in addition, be defined as the amount required to augment the activity of LLL12B. The effective amount of the CDK may alternatively, or in addition, also be defined as the amount required to induce a synergistic effect with LLL12B.

LLL12B and the additional therapeutic agent(s) may be administered in either order, alone or in combination, sequentially or concurrently, with overlapping or non-overlapping periods of administration.

In some aspects of this embodiment, the combination of LLL12B and the additional therapeutic agent(s) has a synergistic therapeutic effect on the cancer. Thus, the therapeutic effect of the combination of LLL12B and the additional therapeutic agent(s) may be greater than the additive effects seen when either LLL12B or the additional therapeutic agent(s) is administered alone.

In one aspect, the invention provides a method of treating TNBC in a subject, comprising administering a therapeutically-effective amount of LLL12B to a subject having TNBC, thereby treating TNBC in a subject.

In another aspect, the invention provides a method of treating pancreatic cancer in a subject, comprising administering a therapeutically-effective amount of LLL12B to a subject having pancreatic cancer, thereby treating pancreatic cancer in a subject.

In a further aspect, the invention provides a method of treating TNBC in a subject, comprising administering a therapeutically-effective amount of LLL12B and one or more PARP inhibitor to a subject having TNBC, thereby treating TNBC in a subject.

In an additional aspect, the invention provides a method of treating pancreatic cancer in a subject, comprising administering a therapeutically-effective amount of LLL12B and one or more PARP inhibitor to a subject having pancreatic cancer, thereby treating pancreatic cancer in a subject.

In another aspect, the invention provides a method of treating TNBC in a subject, comprising administering a therapeutically-effective amount of LLL12B and one or more CDK inhibitor to a subject having TNBC, thereby treating TNBC in a subject.

In yet a further aspect, the invention provides a method of treating pancreatic cancer in a subject, comprising administering a therapeutically-effective amount of LLL12B and one or more CDK inhibitor to a subject having pancreatic cancer, thereby treating pancreatic cancer in a subject.

### Inhibiting STAT3 Activity

The invention is also directed to methods based on the specific activity of LLL12B, i.e. inhibiting STAT3 activity. Thus, the invention is directed to methods of inhibiting STAT3 activity, either in vitro or in vivo.

In one aspect, the invention provides a method of inhibiting STAT3 activity in vitro, comprising contacting cells expressing STAT3 with an amount of LLL12B effective to inhibit STAT3 activity, thereby inhibiting STAT3 activity in vitro.

In a related aspect, the invention provides a method of inhibiting STAT3 activity in vivo, comprising administering to a subject having cells expressing STAT3 an amount of LLL12B effective to inhibit STAT3 activity, thereby inhibiting STAT3 activity in vivo.

The cells expressing STAT3 may be cells of a cancer. Such cancers are those characterized by persistent or constitutive expression (or over expression) of STAT3, and they are defined above. However, exemplary cancers include, but are not limited to, breast cancer, such as triple-negative breast cancer (TNBC), and pancreatic cancer.

The effective amount of LLL12B for in vitro methods is an amount ranging from about 0.25 μM to about 5 μM, culture media.

The effective amount of LLL12B for in vivo methods is an amount ranging from about 1 to about 20 mg/kg, body weight.

In in vitro aspects, the cells may also be contacted with an effective amount of one or more additional therapeutic agents, such as a PARP inhibitor or a CDK inhibitor. The effective amount of PARP inhibitors for in vitro methods is an amount ranging from about 0.01 μM to about 20 μM, culture media. The effective amount of CDK inhibitors for in vitro methods is an amount ranging from about 0.5 μM to about 5 μM, culture media.

In in vivo aspects, an effective amount of one or more additional therapeutic agents, such as a PARP inhibitor or a CDK inhibitor, may also be administered to the subject. The effective amount of PARP inhibitors for in vivo methods is an amount ranging from about 0.05 to about 15 mg/kg, body weight. The effective amount of CDK inhibitors for in vivo methods is an amount ranging from about 10 to about 250 mg/kg, body weight.

Suitable PARP inhibitors for use in these methods are those defined above, but include talazoparib, olaparib, rucaparib, veliparib and niraparib.

Suitable CDK inhibitors for use in these methods are those defined above, but include abemaciclib, palbociclib, ribociclib, Alvocidib, Dinaciclib, P276-00, AT7519 and Roniciclib.

When included, the combination of LLL12B and the one or more additional therapeutic agent may have a synergistic effect on inhibiting STAT3 activity, whether in vitro or in vivo.

### Inhibiting Growth of Cells Expressing STAT3

The invention is directed to further methods based on the specific activity of LLL12B, i.e. inhibiting growth of cells expressing STAT3. Thus, the invention is directed to methods of inhibiting growth of cells expressing STAT3, either in vitro or in vivo.

In one aspect, the invention provides a method of inhibiting growth of cells expressing STAT3 in vitro, comprising contacting cells expressing STAT3 with an amount of LLL12B effective to inhibit growth of the cells, thereby inhibiting growth of cells expressing STAT3 in vitro.

In a related aspect, the invention provides a method of inhibiting growth of cells expressing STAT3 in vivo, comprising administering to a subject having cells expressing STAT3 an amount of LLL12B effective to inhibit growth of the cells, thereby inhibiting growth of cells expressing STAT3 in vivo.

The cells expressing STAT3 may be cells of a cancer. Such cancers are those characterized by persistent or constitutive expression (or over expression) of STAT3, and they are defined above. However, exemplary cancers include, but are not limited to, breast cancer, such as triple-negative breast cancer (TNBC), and pancreatic cancer.

The effective amount of LLL12B for in vitro methods is an amount ranging from about 0.25 μM to about 5 μM, culture media.

The effective amount of LLL12B for in vivo methods is an amount ranging from about 1 to about 20 mg/kg, body weight.

In in vitro aspects, the cells may also be contacted with an effective amount of one or more additional therapeutic agents, such as a PARP inhibitor or a CDK inhibitor. The effective amount of PARP inhibitors for in vitro methods is an amount ranging from about 0.01 μM to about 20 μM, culture media. The effective amount of CDK inhibitors for in vitro methods is an amount ranging from about 0.5 μM to about 5 μM, culture media.

In in vivo aspects, an effective amount of one or more additional therapeutic agents, such as a PARP inhibitor or a CDK inhibitor, may also be administered to the subject. The effective amount of PARP inhibitors for in vivo methods is an amount ranging from about 0.05 to about 15 mg/kg, body weight. The effective amount of CDK inhibitors for in vivo methods is an amount ranging from about 10 to about 250 mg/kg, body weight.

Suitable PARP inhibitors for use in these methods are those defined above, but include talazoparib, olaparib, rucaparib, veliparib and niraparib.

Suitable CDK inhibitors for use in these methods are those defined above, but include abemaciclib, palbociclib, ribociclib, Alvocidib, Dinaciclib, P276-00, AT7519 and Roniciclib.

When included, the combination of LLL12B and the one or more additional therapeutic agent may have a synergistic effect on inhibiting growth of cells, whether in vitro or in vivo.

### Pharmaceutical Compositions

In each of the aspects and embodiments of the invention, LLL12B may be administered to a subject in the form of a pharmaceutical composition comprising LLL12B and one or more pharmaceutically acceptable carriers, excipients and/or diluents. The pharmaceutical composition comprising LLL12B may be formulated for oral delivery, although it should be understood that the invention is not limited to such formulations or related routes of delivery.

The pharmaceutical compositions may be formulated for and administered by, for example, oral, sublingual, intranasal, intraocular, rectal, transdermal, mucosal, pulmonary, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration. In preferred aspects of each of the embodiments on the invention, the pharmaceutical composition is administered to the subject as an oral formulation.

Pharmaceutically acceptable carriers, excipients and diluents are those compounds, solutions, substances or materials that can be used to produce formulations of the LLL12B and/or additional therapeutic agents that are suitable to be administered to a subject, such as a human. In particular, carriers, excipients and diluents of the present invention are those useful in preparing a pharmaceutical composition that is generally safe, non-toxic and neither biologically nor otherwise undesirable and that may present pharmacologically favorable profiles, and includes carriers and diluents that are acceptable for veterinary use as well as human pharmaceutical use. Suitable pharmaceutically acceptable carriers, excipients and diluents are well known in art and can be determined by those of skill in the art as the clinical situation warrants. Examples of suitable carriers and diluents include dextrose, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, a cyclodextrin or a cyclodextrin derivative (including HPCD ((2-hydroxypropyl)-cyclodextrin) and (2-hydroxyethyl)-cyclodextrin), hydrophilic and hydrophobic carriers, and combinations thereof. Hydrophobic carriers include, for example, fat emulsions, lipids, PEGylated phospholipids, polymer matrices, biocompatible polymers, lipospheres, vesicles, particles, and liposomes. The terms specifically exclude cell culture medium. More particularly: (1) 5% (w/v) dextrose, or (2) water (e.g., sterile water; Water-For-Injection), may be used as a pharmaceutically acceptable carrier. Pharmaceutically acceptable diluents also include tonicity agents that make the composition compatible with blood. Tonicity agents are particularly desirable in injectable formulations.

Excipients included in a formulation have different purposes depending, for example on the nature of the drug, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweeteners, perfuming agents, flavoring agents, coloring agents, administration aids, and combinations thereof.

The pharmaceutical compositions may contain common carriers and excipients, such as cornstarch or gelatin, lactose, sucrose, microcrystalline cellulose, kaolin, mannitol, dicalcium phosphate, sodium chloride, alginic acid, croscarmellose sodium, and sodium starch glycolate.

The particular carrier, diluent or excipient used will depend upon the means and purpose for which the active ingredient is being applied.

Acceptable methods for preparing the pharmaceutical compositions according to the invention are known to those skilled in the art. For example, pharmaceutical compositions may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

### Methods of Treatment

The methods of the invention include methods of treating cancer in a subject comprising administering therapeutically-effective amounts of LLL12B and, optionally, one or more additional therapeutic agents, such as PARP inhibitors and/or CDK inhibitors, to a subject having cancer. When both are used, LLL12B and the additional therapeutic agents may be administered in any order, separately or in combination (two drugs per combination, or three drugs per combination, or four or more drugs per combination), sequentially or concurrently, with overlapping or non-overlapping periods of administration. The methods of the invention thus include methods of treating cancer in a subject, comprising concurrently administering therapeutically effective amounts of LLL12B and one or more additional therapeutic agents to a subject having cancer. The methods of the invention thus also include methods of treating cancer in a subject, comprising sequentially administering therapeutically effective amounts of LLL12B and one or more additional therapeutic agents to a subject having cancer.

The terms “treating” and “treatment” mean at least the mitigation of cancer, or a disease condition or symptom associated with cancer in a subject that is achieved by a reduction of growth, replication, and/or propagation, or death or destruction of cancer and/or cancer cells, on or in the subject. The terms “treating” and “treatment” include curing, healing, inhibiting, relieving from, improving and/or alleviating, in whole or in part, the cancer or associated disease condition or symptom. The mitigation of cancer or associated disease condition or symptom may be about 100%, 99%, 98%, 97%, 96%, 95%, 90%, 80%, 70%, 60%, 50%, 40%, 30%, 20%, 10%, 5% or 1% in the subject, versus a subject to which LLL12B and, optionally, additional therapeutic agents taught herein have not been administered. In one aspect, treating means reducing the population of cancer cells causing the cancer in the subject to an undetectable level, where detection is by any conventional means, such assay a blood sample in the laboratory. In another aspect, treating means complete healing of the cancer, shown by an absence of clinical symptoms associated with the cancer. In a further aspect of the invention, treating means the mitigation of cancer or an associated disease condition or symptom by at least about 90% in the subject. In an additional aspect, treating means the mitigation of cancer or an associated disease condition or symptom by at least about 95% in the subject.

The methods of the invention include also methods of prolonging survival of a subject having cancer comprising administering therapeutically effective-amounts of LLL12B and one or more additional therapeutic agents to a subject having cancer. The LLL12B and the additional therapeutic agents may be administered in any order, separately or in combination (two drugs per combination, or three drugs per combination, or four or more drugs per combination), sequentially or concurrently, with overlapping or non-overlapping periods of administration. The methods of the invention thus include methods of prolonging survival of a subject having cancer, comprising concurrently administering therapeutically effective amounts of LLL12B and one or more additional therapeutic agents to a subject having cancer. The methods of the invention thus also include methods of prolonging survival of a subject having cancer, comprising sequentially administering therapeutically effective amounts of LLL12B and one or more additional therapeutic agents to a subject having cancer.

The term “prolonging survival” means extending the life span of a subject having cancer by at least one day versus a subject having the same cancer that does not receive the LLL12B and one or more additional therapeutic agents. Prolonged survival includes increasing the life span of the subject by at least: 1, 2, 3, 4 or more weeks, 1, 2, 3, 4, 5, 6, 7, 8, 9, 10, 11, 12 or more months, or 1, 2, 3, 4, 5, or more years.

The amount of the drugs sufficient to have an effect on cancer (additive, synergistic or otherwise) in a subject will vary, for example, in view of the identity of the drugs being used in the combination, the physical characteristics of the subject, the severity of the subject's symptoms, the form of the cancer, the identity of the cancer, the formulations and means used to administer the drugs, and the method being practiced. The specific dose for a given subject is usually set by the judgment of the attending physician. However, each dose comprising LLL12B is typically between about 0.1 to about 50 mg/kg body weight, or about 1 to about 20 mg/kg body weight, or about 2 to about 10 mg/kg body weight. In each dose comprising a PARP inhibitor, the amount of the drug is typically between about 0.01 to about 25 mg/kg body weight, or about 0.05 to about 15 mg/kg body weight, or about 0.1 to about 10 mg/kg body weight. In each dose comprising a CDK inhibitor, the amount of the drug is typically between about 5 to about 500 mg/kg body weight, or about 10 to about 250 mg/kg body weight, or about 50 to about 100 mg/kg body weight.

Depending on the means of administration, the dose may be administered all at once, such as with an oral formulation in a capsule, or slowly over a period of time, such as with an intravenous administration. For slower means of administration, the administering period can be a matter of minutes, such as about 5, 10, 15, 20, 25, 30, 35, 40, 45, 50, 55, 60, 65, 70, 75, 80, 85, 90, 95, 100, 105, 110, 115, 120 or more minutes, or a period of hours, such as about 0.5, 1, 1.5, 2, 2.5, 3, 3.5, 4, 4.5, 5 or more hours. The administration of the dose may be interrupted, such as where the dose is administered via intravenous infusion and the dose is divided into two or more infusion bags. Under such circumstances, the administration of the dose may be interrupted while the infusion bags are changed.

As used herein, the terms “dose”, “unit dose”, “dosage”, “effective dose” and related terms refer to physically discrete units that contain a predetermined quantity of active ingredient or therapeutic agent (drug) calculated to produce a desired therapeutic effect. A single dose is thus a predetermined quantity of an artemisinin and/or one or more additional therapeutic agent that is administered to a subject.

As used herein, a “subject” is a human, a non-human primate, bird, horse, cow, goat, sheep, a companion animal, such as a dog, cat or rodent, or other mammal.

### Additive and Synergistic Effects

In many instances, the combinations of drugs taught herein, i.e., the combinations of LLL12B and one or more additional therapeutic agents, such as PARP inhibitors and/or CDK inhibitor, target two or more different aspects of a cancer cell, such as two or more different structures, or two or more different pathways, or one or more structures on one hand and one or more pathways on the other. As a result, while the combinations of LLL12B and one or more additional therapeutic agents may have an additive therapeutic effect on a cancer, the combinations may also or alternatively have a synergistic therapeutic effect on the cancer. Synergistic therapeutic effects are those that are substantially greater than what is seen when cancer cells are treated with either drug alone.

### III. Examples

### Experiment #1-LLL12B Activity

### Materials and Methods

Cell culture and reagents. Human breast cancer cell lines T47D, MDA-MB-231, SUM159, and murine TNBC cell line 4T1 were used in this study. Cells were cultured in Dulbecco's Modified Eagle Medium (Corning, NY, USA) supplemented with 10% fetal bovine serum (Sigma-Aldrich, St. Louis, MO, USA) and 1% Penicillin/Streptomycin (Sigma-Aldrich, St. Louis, MO, USA). All cells were maintained at 37° C. in a humidified atmosphere with 5% CO₂.

LLL12B was synthesized by the laboratory of Dr. Chenglong Li at the University of Florida College of Pharmacy and C188-9 was purchased from MedChemExpress LLC (Monmouth Junction, NJ, USA). LLL12B and C188-9 were dissolved in sterile dimethyl sulfoxide (DMSO). Human Interleukin-6 (IL-6), Interferon-γ (IFN-γ), and Epidermal Growth Factor (EGF) were prepared according to the manufacturer's instructions (Cell Signaling Technology, Danvers, MA, USA).

SiRNA and transfection. STAT3 siRNA and Negative Control siRNA (Cell Signaling Technology, Danvers, MA, USA) were used to knock down STAT3 in MDA-MB-231, SUM159, and 4T1 cell lines. Cells were transfected with STAT3 siRNA or Negative Control siRNA using Lipofectamine RNAiMAX (Invitrogen, Carlsbad, CA, USA) according to the manufacturer's instructions. After 72 hours, cell viability was examined by MTT cell viability assay, and the transfection efficiency was examined by western blot.

MTT cell viability assay. MDA-MB-231, SUM159, and 4T1 cells were plated in a 96-well plate in triplicate. After overnight incubation, cells were transfected with STAT3 siRNA or Negative Control siRNA for 72 hours. Subsequently, 20 μL of 5 mg/ml thiazolyl blue tetrazolium dye solution (Sigma-Aldrich, St. Louis, MO, USA) was added to each well of the plate and incubated at 37° C. for 4 hours. 100 μL of N, N-dimethylformamide solution (Sigma-Aldrich, St. Louis, MO, USA) was used to dissolve the formazan. Absorbance at 595 nm was read using an EL808 Ultra Microplate Reader (BioTek, Winooski, VT, USA).

Western blot. Cells were collected and total protein was extracted using cell lysis buffer (Cell Signaling Technology, Danvers, MA, USA). Protein concentration was determined using the Pierce BCA Protein Assay Kit (Thermo Fisher Scientific, Waltham, MA, USA) according to the manufacturer's instructions. Equal amounts of protein were separated by 10% SDS-PAGE gels and transferred to PVDF membranes. Membranes were blocked in 5% non-fat milk at room temperature and probed with specific antibodies P-STAT3 (Y705), STAT3, Cyclin D1, Cleaved Caspase-3, P-STAT1 (Y701), STAT1, P-ERK, ERK or GAPDH (1:1000, Cell Signaling Technology, Danvers, MA, USA) at 4° C. overnight. The blots were visualized using SuperSignal™ West Femto Maximum Sensitivity Substrate (Thermo Fisher Scientific, Waltham, MA, USA) and an Amersham Imager 680 (GE Healthcare Life Sciences, Marlborough, MA, USA) after incubating with horseradish peroxidase (HRP)-conjugated anti-rabbit secondary antibody (1:5000, Cell Signaling Technology, Danvers, MA, USA).

Immunofluorescence staining. SUM159 cells were seeded in a 6-well plate and treated with LLL12B or DMSO for 8 hours. Cells were fixed with 4% paraformaldehyde (PFA) for 15 minutes at room temperature and then permeabilized with ice-cold 100% methanol for 10 minutes at −20° C. After blocking in 3% Bovine Serum Albumin (BSA)/0.1% Triton X-100, cells were incubated with rabbit anti-Phospho-Stat3 (Tyr705) (1:100, Cell Signaling Technology, Danvers, MA, USA) overnight at 4° C., then with secondary antibody for 1 hour at room temperature. Cells were photographed using a Leica fluorescence microscope (Leica Microsystems Inc, Deerfield, IL, USA).

Caspase-3/7 activity assay. The Caspase-3/7 Fluorescence Assay Kit (Cayman, Ann Arbor, MI, USA) was used to detect the activation of caspase-3/7 and the assay was performed according to the manufacturer's instructions. Briefly, MDA-MB-231, SUM159, and 4T1 cells were seeded in a 96-well plate in triplicate. After overnight incubation, cells were treated with either LLL12B or DMSO for 5 hours. Cells were lysed and processed to measure caspase-3/7 activity and the fluorescence intensity was read (excitation=485 nm; emission=535 nm).

Flow cytometry analysis. Flow cytometry was performed to detect cell apoptosis. The APC Annexin V Apoptosis Detection Kit (Biolegend, San Diego, CA, USA) was used for Annexin V staining. Cells were seeded in a 35 mm culture dish at a density of 1×10⁵ cells/dish. After overnight incubation, cells were treated with DMSO or LLL12B for 8-12 hours. Cells were harvested and stained with Annexin V-APC and propidium iodide (PI) according to the manufacturer's instructions. After staining, cells were counted using a FACSCanto II flow cytometer (BD Biosciences, San Jose, CA, USA).

Colony formation assay. MDA-MB-231, SUM159, and 4T1 cells were seeded in a 6-well plate at a density of 1000 cells/well and treated with DMSO or LLL12B for 24 hours, and then cultured in a drug-free culture medium for 7 days. Colonies were fixed with methanol for 30 min and stained with 1% crystal violet in 25% methanol for 2 hours at room temperature.

Wound healing assay. MDA-MB-231, SUM159, and 4T1 cells were seeded in a 6-well plate and cultured until fully confluent. A straight scratch crossing the monolayer was created using a 200 μl pipette tip and images of scratched areas were taken. Next, cells were treated with DMSO or LLL12B. Then, images were taken until the scratch of DMSO-treated cells was closed and the relative migration (%) was calculated.

Orthotopic mammary fat pad tumor model in vivo. All mice were used under guidelines approved by the IACUC of University of Maryland School of Medicine. 6-week-old female athymic nude mice were purchased from The Jackson Laboratory (Bar Harbor, ME, USA). 2.5×10⁶ MDA-MB-231 cells in 50 μl matrigel (BD Science, Franklin Lakes, NJ, USA) were inoculated into the 4th mammary fat pads bilaterally. After the tumor volume reached approximately 50 mm³, mice were randomly divided into two groups (3 mice with 6 tumors in each group). LLL12B (2.5 mg/kg) or DMSO vehicle was administered daily by oral gavage for 28 days. Tumor size was measured by the length (L) and width (W) using a caliper ruler every 2 days. Tumor volume was calculated using 0.52×L×W². Body weight was monitored every 2 days. At the end of the experiment, tumors were removed and weighed. Part of the tumor tissue was lysed to detect the expression of P-STAT3 (Y705), STAT3, and Cyclin DI using Western blot analysis.

Statistical analysis. The statistical significance (P-value) between the control and experimental groups was determined by Student's unpaired t-test (two-group comparison) or one-way ANOVA (three-group comparison), and P<0.05 was considered significant. All experiments were carried out in triplicate and data was analyzed using GraphPad software (GraphPad Software Inc, San Diego, CA, USA).

### Results

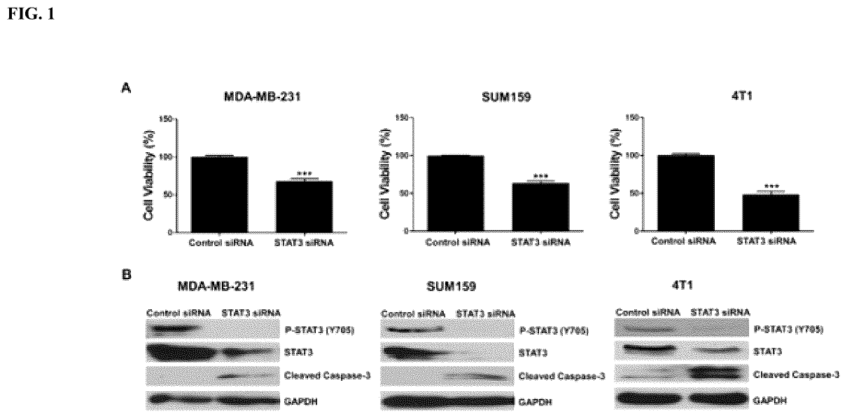

Knockdown of STAT3 inhibits TNBC cell viability. STAT3 is persistently activated in TNBC cells. To explore the roles of STAT3, STAT3 expression was knocked down by STAT3-specific siRNA in human TNBC cell lines MDA-MB-231 and SUM159, and murine TNBC cell line 4T1. MTT assays showed that cell viability was decreased by STAT3 knockdown in MDA-MB-231, SUM159, and 4T1 TNBC cells compared to cells transfected with control siRNA (FIG. 1A). STAT3 knockdown efficiency was evaluated by western blot analysis. As shown in FIG. 1B, STAT3 siRNA reduced phosphorylation of STAT3 (Y705) and total STAT3 expression, and induced expression of the apoptosis marker, Cleaved Caspase-3, in MDA-MB-231, SUM159, and 4T1 cell lines. Hence, the knockdown of STAT3 supports the role of STAT3 in maintaining cell viability in TNBC cells.

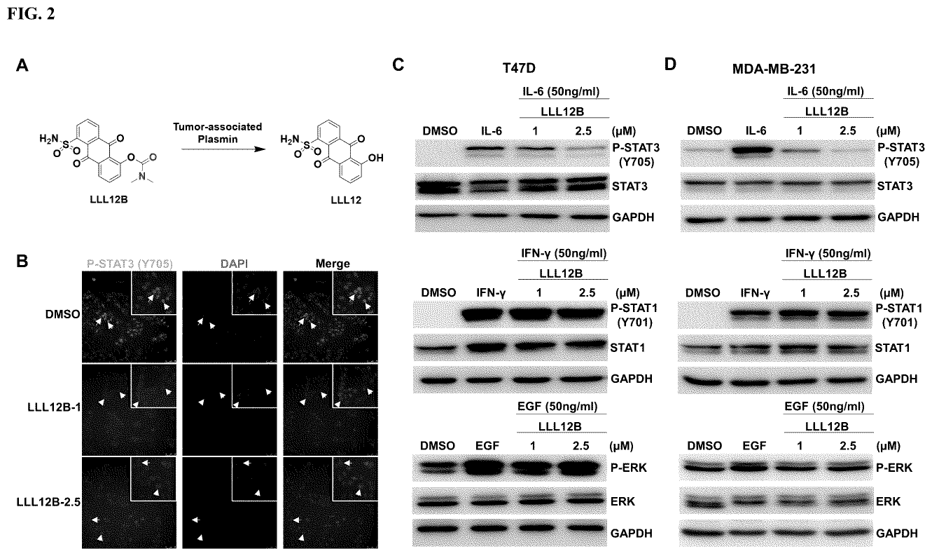

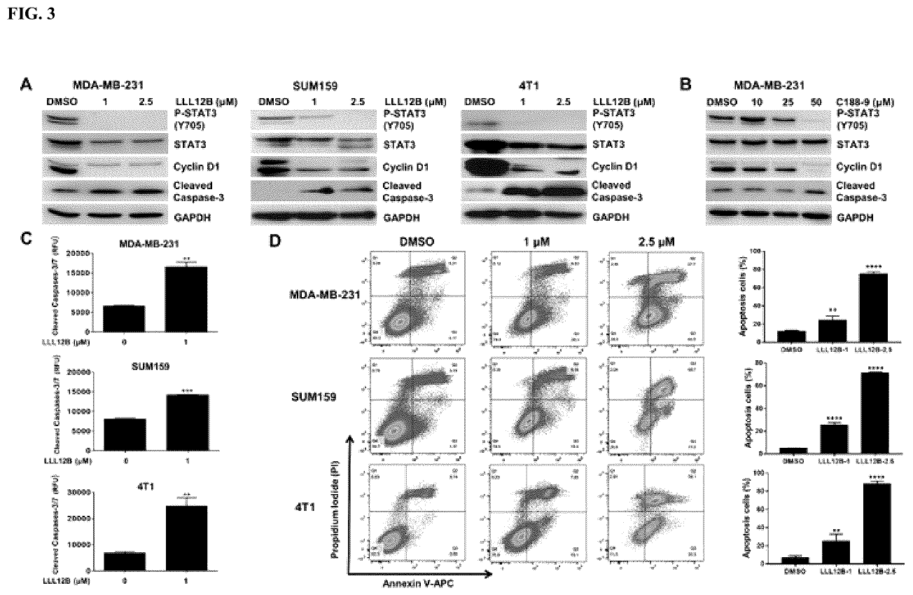

LLL12B inhibits STAT3 nuclear translocation and blocks IL-6 induced STAT3 phosphorylation. Carbamate esters are one of the prodrug types which are developed to improve the pharmacokinetic properties of drugs [29,30]. A carbamate prodrug of STAT3 inhibitor LLL12, named LLL12B which is activated by the tumor-associated plasmin, was designed and tested in this study (FIG. 2A). The nuclear translocation of phosphorylated STAT3 (Y705) is required for regulating the transcription of STAT3 target genes. Thus, the cellular localization of P-STAT3 (Y705) in SUM159 cells with LLL12B treatment was examined using immunofluorescence staining. In DMSO-treated SUM159 cells, P-STAT3 (Y705) was mainly present in the nucleus. However, the distribution of P-STAT3 (Y705) in the nucleus was blocked by LLL12B treatment (FIG. 2B), supporting that STAT3 inhibitor LLL12B was activated in the SUM159 cells. It has been shown that LLL12B inhibited IL-6 induced STAT3 phosphorylation in human medulloblastoma cells [25,31]. To further confirm the selectivity of LLL12B in breast cancer cells, its effects was tested on the phosphorylation of STAT3, STAT1, or ERK following the stimulation with IL-6, IFN-γ, or EGF in T47D breast cancer cells which express lower basal levels of P-STAT3 (FIG. 2C), and MDA-MB-231 TNBC cells (FIG. 2D). As shown by Western blot, IL-6 induced phosphorylation of STAT3 (Y705) and the induction of P-STAT3 was inhibited by LLL12B in both T47D and MDA-MB-231 cell lines. However, LLL12B did not inhibit the induction of P-STAT1 (Y701) which is stimulated by IFN-γ in T47D and MDA-MB-231 cell lines, or the induction of P-ERK which is stimulated by EGF in T47D cells. There was a slight induction of P-ERK stimulated by EGF in MDA-MB-231 cells, and LLL12B had little effect on the inhibition of P-ERK. Collectively, these results confirm that LLL12B selectively inhibits the IL-6/STAT3 signaling pathway.

LLL12B inhibits STAT3 activation and induces TNBC cell apoptosis. To explore the activity of LLL12B in TNBC cells, MDA-MB-231, SUM159, and 4T1 cell lines were treated with LLL12B and the vehicle control DMSO. As shown in FIG. 3A, Western blot analysis demonstrated that the phosphorylation of STAT3 and its downstream target Cyclin DI were decreased in LLL12B-treated cells compared to DMSO-treated cells. Additionally, the expression of Cleaved Caspase-3 was enhanced in LLL12B treated cells. As a comparison, MDA-MB-231 cells were treated with the oral STAT3 inhibitor C188-9, also named TTI-101, which is currently in phase I clinical study for advanced solid tumors [32]. As shown in FIG. 3A, 1 μM of LLL12B effectively inhibited P-STAT3, and the STAT3 downstream target Cyclin D1, as well as induced Cleaved Caspase-3 in MDA-MB-231 cells, while 25 μM and 50 μM of C188-9 were needed to inhibit P-STAT3 and Cyclin DI (FIG. 3B). In conjunction with Western blot analysis, the caspase-3/7 activity assay demonstrated that LLL12B treatment induced the activation of caspase-3/7 in MDA-MB-231, SUM159, and 4T1 cell lines (FIG. 3C). To further confirm cell apoptosis, annexin V and PI staining was performed using flow cytometry. As shown in FIG. 3D, the percentage of apoptotic cells was significantly increased in LLL12B-treated cells compared to DMSO-treated cells. Overall, these results indicate that LLL12B is a potent STAT3 inhibitor and induces apoptosis in TNBC cells.

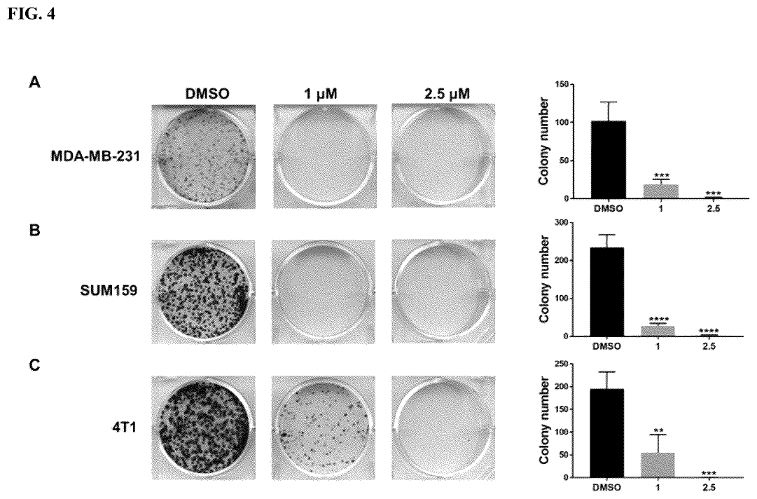

LLL12B inhibits cell colony formation in TNBC cells. To investigate the longer-term effect of LLL12B on TNBC cell expansion, the colony formation assay was performed. MDA-MB-231, SUM159, and 4T1 cells were treated with LLL12B or DMSO for 24 hours and recovered in a drug-free culture medium for 7 days. As shown in FIG. 4, the LLL12B treatment showed a significant reduction in colony numbers as compared to the DMSO treatment.

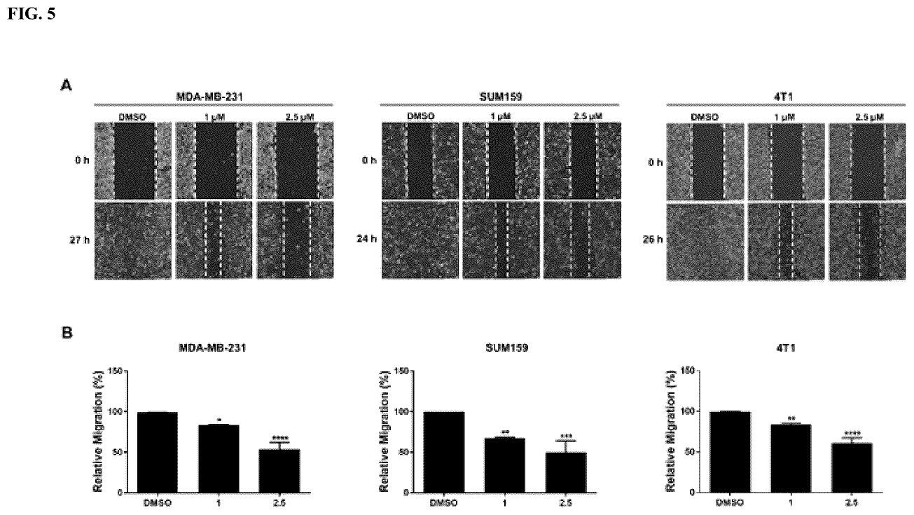

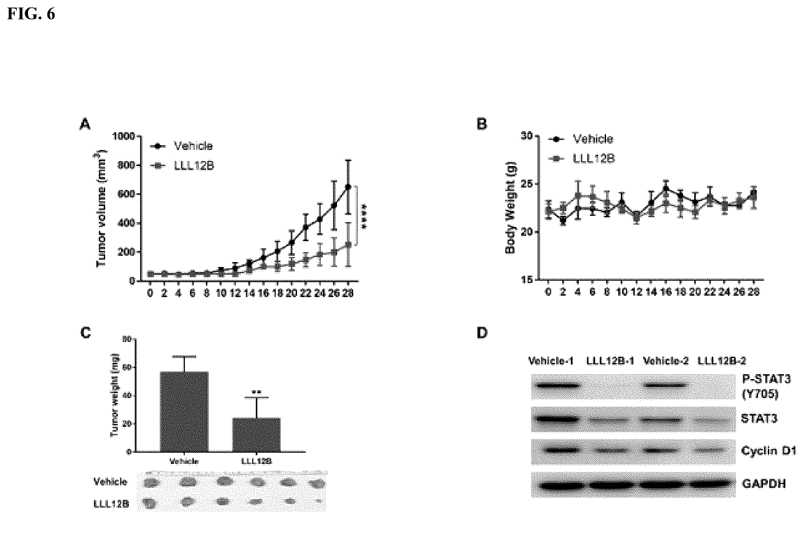

LLL12B inhibits cell migration in TNBC cells. Because cancer cell migration is relevant to their ability to metastasize, the inhibitory effect of LLL12B on cell migration was determined by performing a wound healing assay in MDA-MB-231, SUM159, and 4T1 cells. Notably, LLL12B treatment, compared to DMSO treatment, effectively inhibited wound closure in MDA-MB-231, SUM159, and 4T1 cells (FIG. 5). Therefore, these results provide experimental evidence that LLL12B is capable of inhibiting the migration of TNBC cells.

LLL12B suppressed tumor growth in the MDA-MB-231 orthotopic tumor model in vivo. Female nude mice were implanted with MDA-MB-231 cells to establish an orthotopic tumor model to further evaluate LLL12B efficacy in vivo. MDA-MB-231 cells were inoculated into the 4th mammary fat pads and tumors were allowed to develop until the volume reached approximately 50 mm³. Vehicle control or LLL12B (2.5 mg/kg) was administered once daily by oral gavage for 28 days. Compared to the vehicle-treated group, the tumor volume in the LLL12B treated group was significantly reduced (FIG. 6A). In addition, the LLL12B treated group showed reduced tumor weight (FIG. 6C). There was no obvious reduction of body weights observed in the LLL12B treated group compared to the vehicle-treated group (FIG. 6B) suggesting little acute toxicity. As expected, Western blot analysis of tumor tissues showed that LLL12B suppressed phosphorylation of STAT3 (Y705) and Cyclin DI expression (FIG. 6D). Collectively, these results indicate that LLL12B, as a single agent, can suppress MDA-MB-231 tumor growth in vivo.

The results presented here demonstrate that targeting STAT3 with LLL12B induces apoptosis, inhibits colony formation and cell migration in vitro, and suppresses tumor growth of TNBC cells in vivo. These results support that LLL12B is a potential therapeutic agent either to be used as monotherapy or in combination with another therapeutic agent for TNBC treatment.

### Experiment #2-LLL12B and Pancreatic Cancer

An additional experiment was conducted to determine whether LLL12B has activity against pancreatic cancer cells.

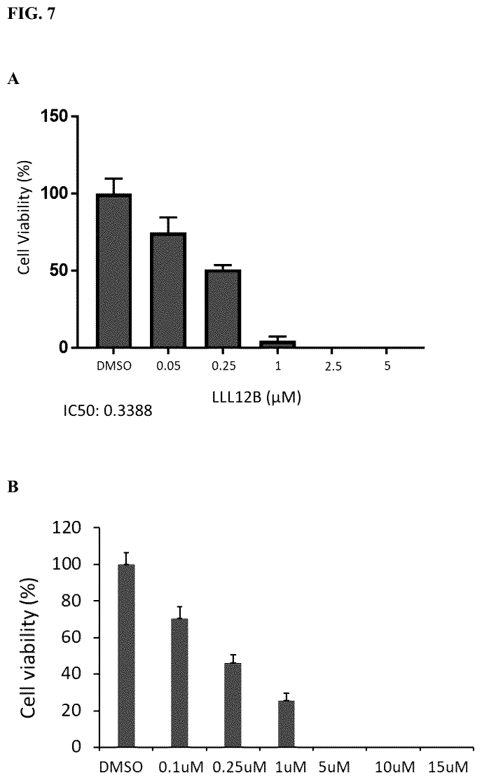

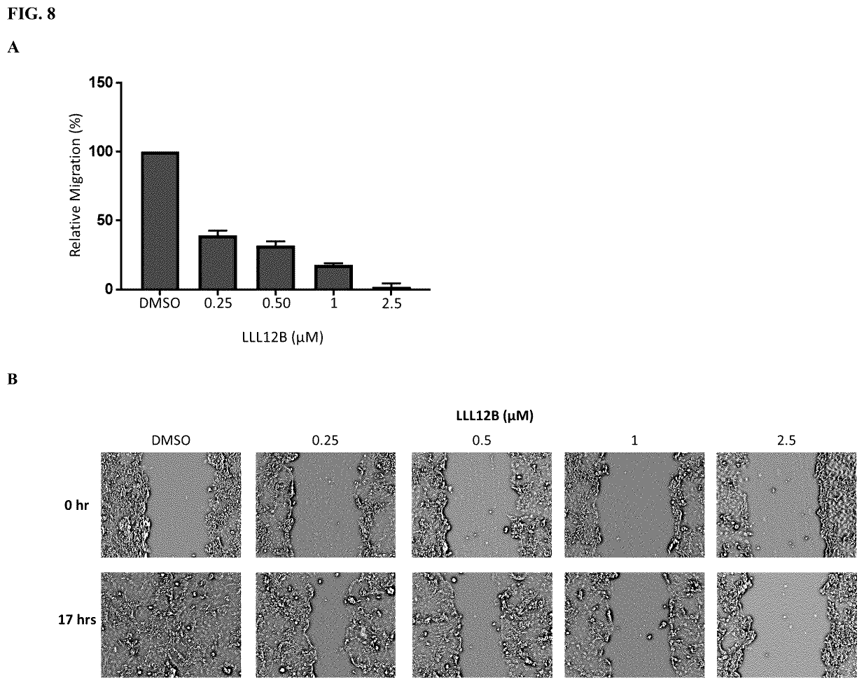

The human pancreatic cancer cell lines HPAC and PANC-1 were seeded and allowed to attach for 24 hours. Then cells were treated with different concentrations of LLL12B for 3 days and the cell viability was determined by MTT cell viability assay. As shown in FIG. 7, LLL12B significantly inhibits cell viability of human pancreatic cancer cell lines HPAC (A) and PANC-1 (B).

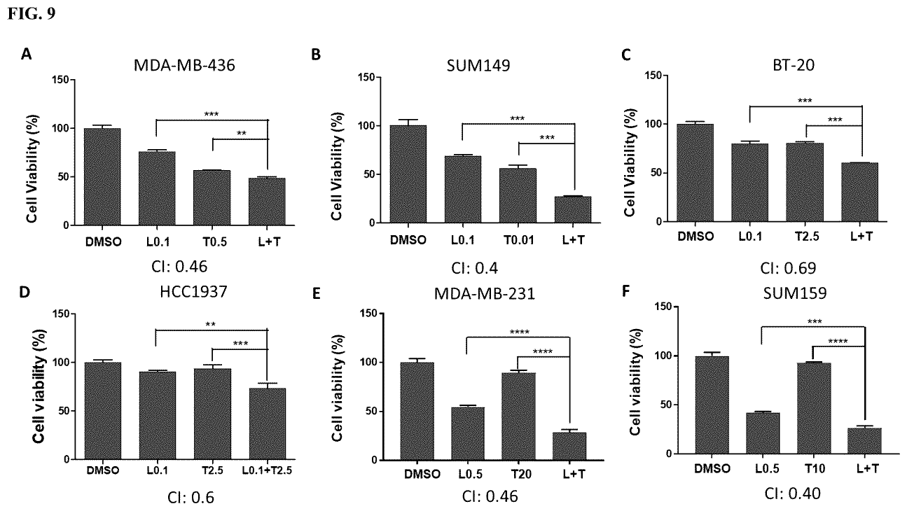

The inhibitory effects on cell migration by LLL12B in pancreatic cancer cells was also examined. The human HPAC pancreatic cancer cells were seeded and allowed to attach for 24 hours. Then cells were treated with different concentrations of LLL12B for approximate 24 hours and the cell migration was determined by wound healing assay. As shown in FIG. 8, LLL12B significantly inhibits cell migration of human pancreatic cancer cell line HPAC, with percent migration shown graphically (A) and via microscopic studies (B). These results support the potential utility of LLL12B as a therapeutic agent in human pancreatic cancer

Experiment #3-LLL12B in Combination with Talazoparib

To examine the synergy between STAT3 and PARP inhibition, human triple-negative breast cancer cell lines MDA-MB-436, SUM149, BT-20, HCC1937, MDA-MB-231 and SUM159 were treated with LLL12B in combination with PARP inhibitor Talazoparib.

As shown in FIG. 9(A-F), the drug combination showed a greater inhibition than single drug treatment. The combination index (CI) values are below 1, indicating the combination of LLL12B and Talazoparib was synergistic. Cells were treated with LLL12B and Talazoparib for 3 days and the cell viability was determined by MTT assay. (L: LLL12B; T: Talazoparib; L0.1:0.1 μM LLL12B; L0.5:0.5 μM LLL12B; T0.5:0.5 μM Talazoparib; T0.01:0.01 μM Talazoparib; T2.5:2.5 μM Talazoparib; T20: 20 μM Talazoparib; T10: 10 μM Talazoparib).

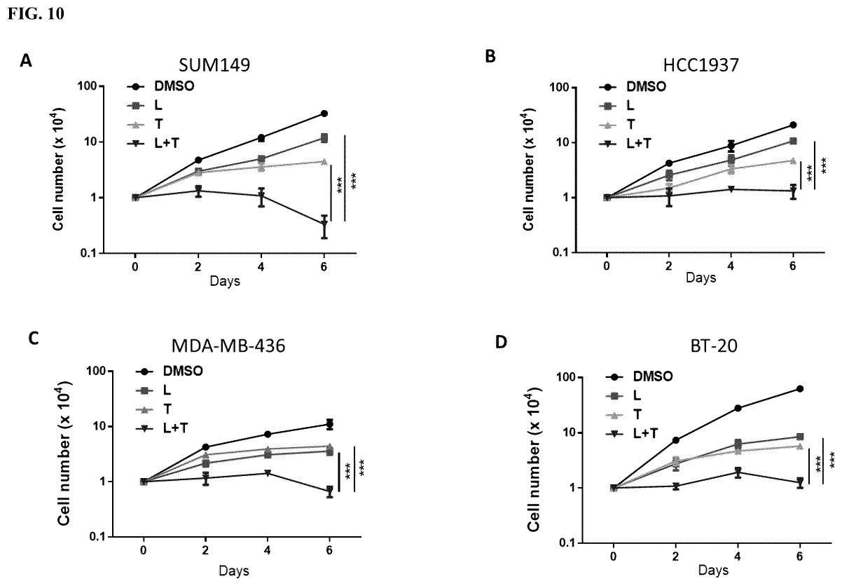

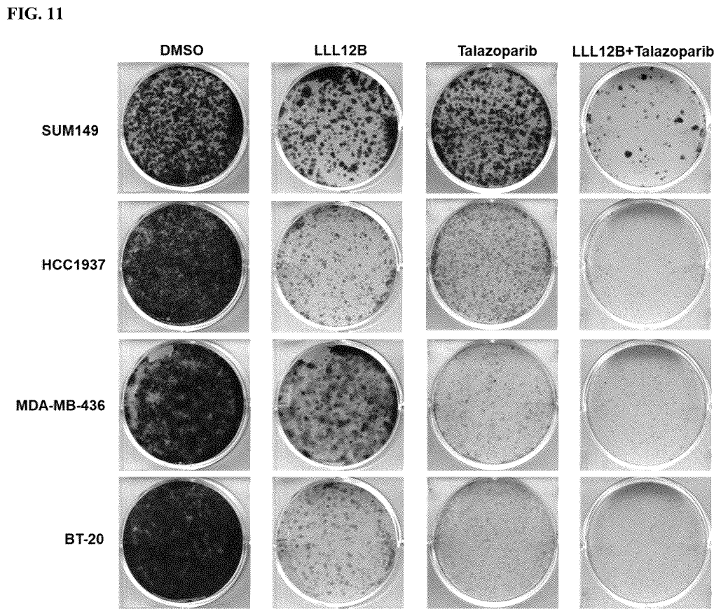

Cell growth curve showed LLL12B in combination with Talazoparib significantly inhibited cell growth in human triple-negative breast cancer cell lines SUM149, HCC1937, MDA-MB-436, and BT-20 (FIG. 10A-D). (L: LLL12B; T: Talazoparib). Furthermore, the combination of LLL12B and Talazoparib significantly inhibited colony formation compared to single drug treatment in human triple-negative breast cancer cell lines SUM149, HCC1937, MDA-MB-436, and BT-20 (FIG. 11).

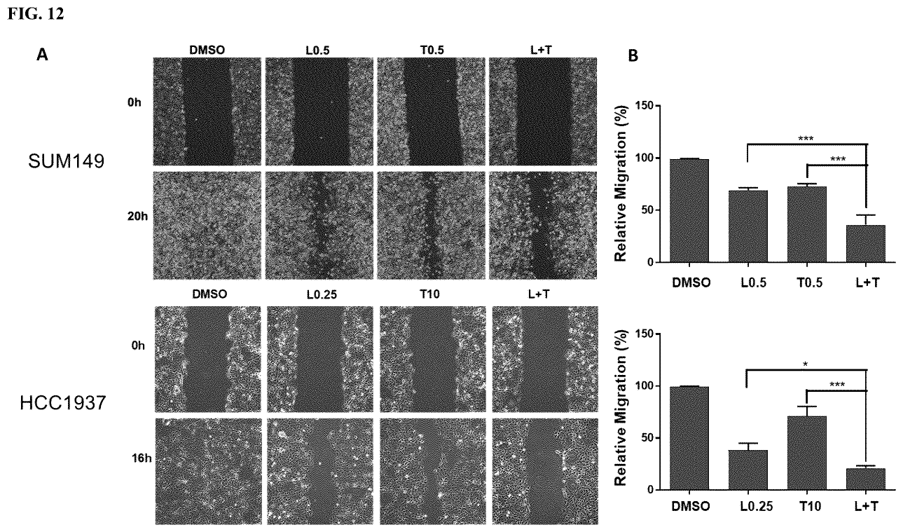

Given the relevance between cancer cell migration and metastasis, cell migration was evaluated using wound healing assay following LLL12B and Talazoparib treatment. As shown in FIGS. 12-14, the inhibition of cell migration was significantly greater with combination treatment compared to single drug treatment in SUM149, HCC1937, MDA-MB-436, BT-20, MDA-MB-231, and SUM159 cell lines. These date indicate that STAT3 inhibitor LLL12B in combination with PARP inhibitor Talazoparib synergistically inhibited cell migration of human triple-negative breast cancer cells.

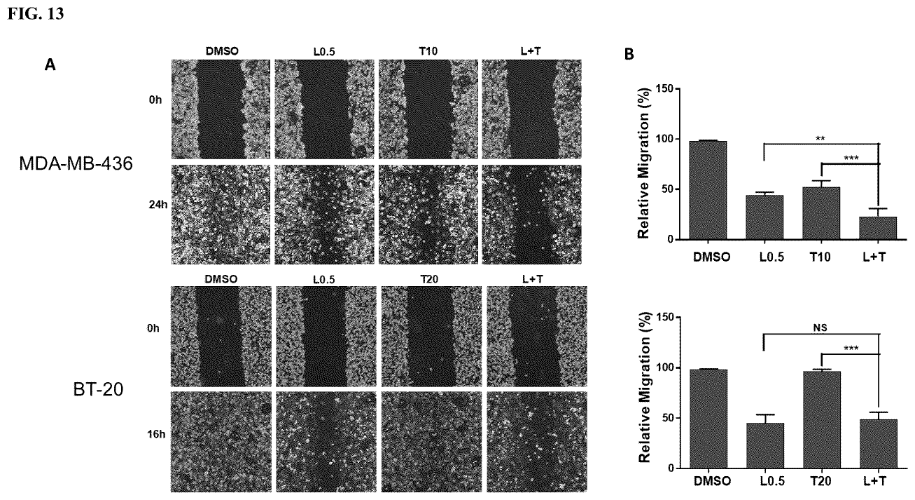

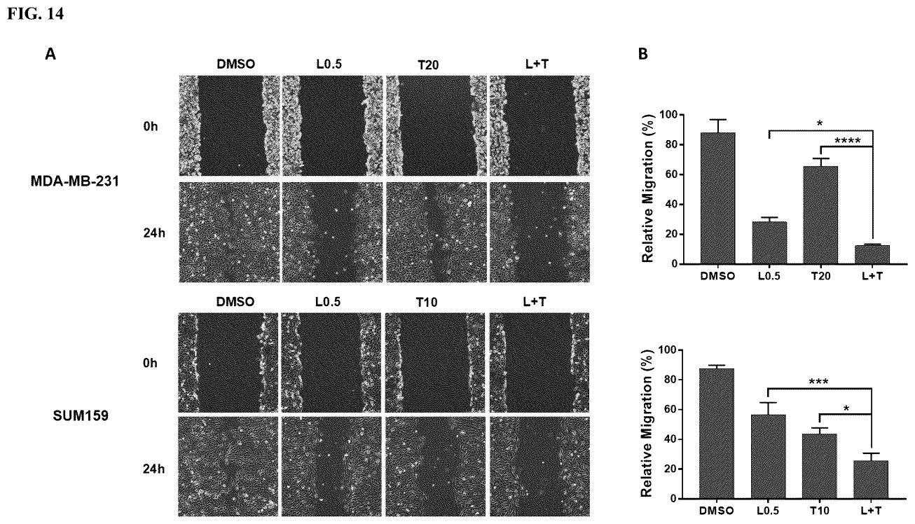

In FIG. 12, L: LLL12B; T: Talazoparib; L0.5:0.5 μM LLL12B; L0.25:0.25 μM LLL12B; T0.5:0.5 μM Talazoparib; T10: 10 μM Talazoparib. In FIG. 13, L: LLL12B; T: Talazoparib; L0.5:0.5μ M LLL12B; T10: 10μ M Talazoparib; T20: 20μ M Talazoparib. In FIG. 14, L: LLL12B; T: Talazoparib; L0.5:0.5μ M LLL12B; T10: 10μ M Talazoparib; T20: 20μ M Talazoparib.

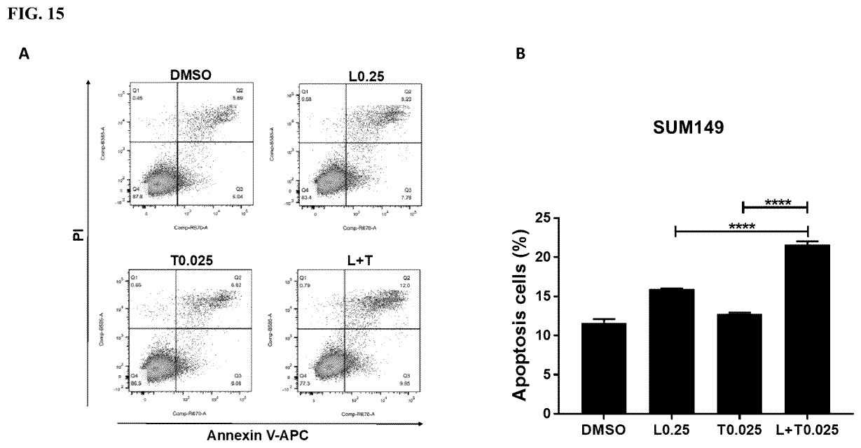

To dissect the mechanism of enhanced cytotoxicity with LLL12B and Talazoparib, cell apoptosis was detected using Annexin V/PI staining performed by flow cytometry. As shown in FIG. 15A-B there were more apoptotic cells with combination treatment compared to single drug treatment in SUM149 cells. L: LLL12B; T: Talazoparib; L0.25:0.25 μM LLL12B; T0.025:0.025 μM Talazoparib.

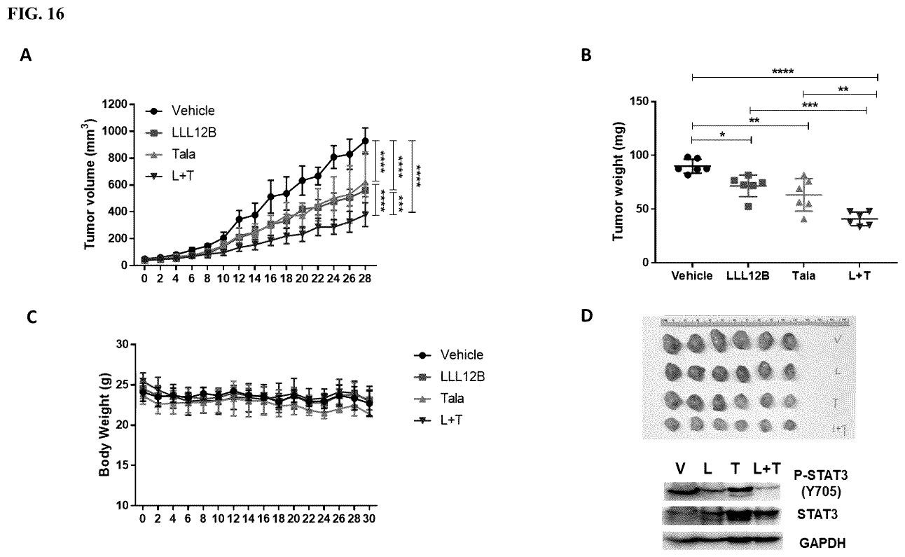

Next, the efficacy of LLL12B and Talazoparib was examined in the SUM149 xenograft model in vivo. As shown in FIG. 16, LLL12B and Talazoparib treatment significantly suppressed tumor growth (FIG. 16A) and reduced tumor weight (FIG. 16B), without body weight change (FIG. 16C). V: Vehicle; L: LLL12B; T: Talazoparib. Western blot analysis further confirmed the inhibition of p-STAT3 in LLL12B and combination treated tumors (FIG. 16D).

Experiment #4-LLL12B in Combination with Abemaciclib

To examine the synergy between STAT3 and CDK4/6 inhibition, human triple-negative breast cancer cell lines MDA-MB-231, SUM159, MDA-MB-231 BoneM and MDA-MB-231 BRM were treated with LLL12B in combination with CDK4/6 inhibitor Abemaciclib.

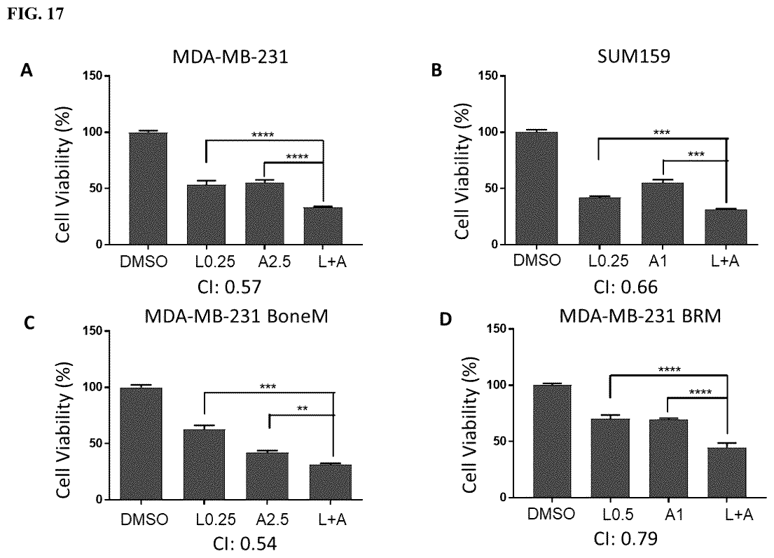

As shown in FIG. 17A-D, LLL12B in combination with Abemaciclib showed a greater inhibition than single drug treatment. The combination index (CI) values are below 1, indicating the combination of LLL12B and Abemaciclib was synergistic. L: LLL12B; A: Abemaciclib; L0.25:0.25 μM LLL12B; L0.5:0.5 μM LLL12B; A2.5:2.5 μM Abemaciclib; A1: 1 μM Abemaciclib.

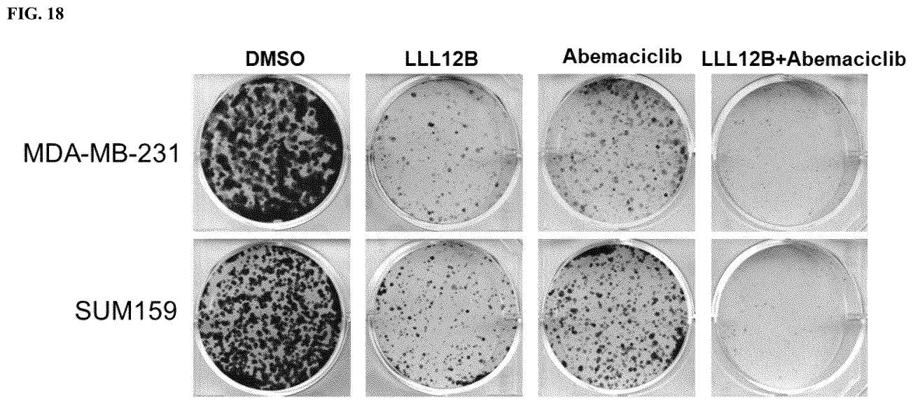

Furthermore, colony formation assay showed the combination of LLL12B and Abemaciclib significantly inhibited colony formation compared to single drug treatment in human triple-negative breast cancer cell lines MDA-MB-231 and SUM159 (FIG. 18).

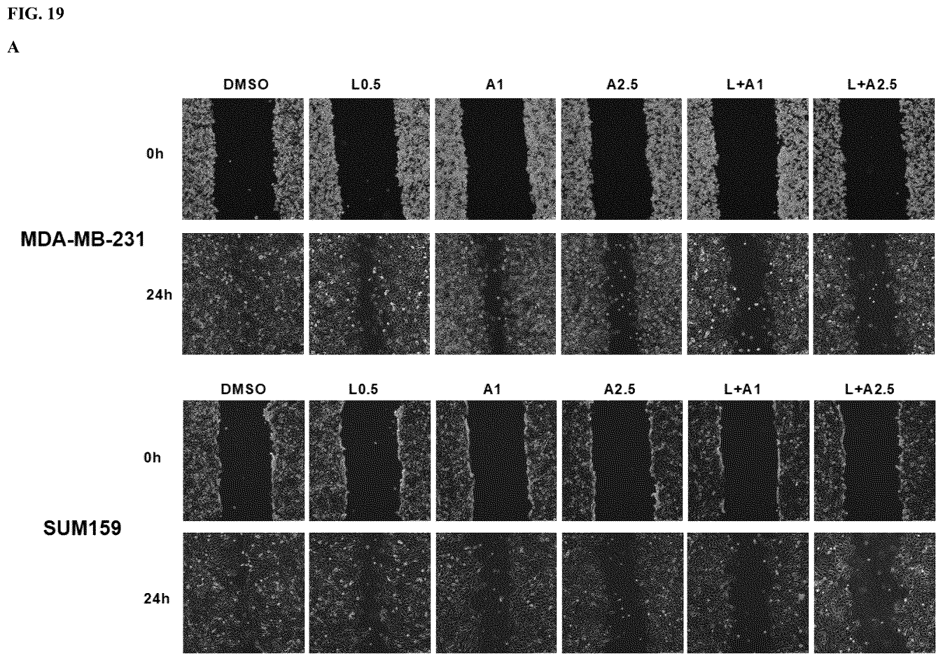

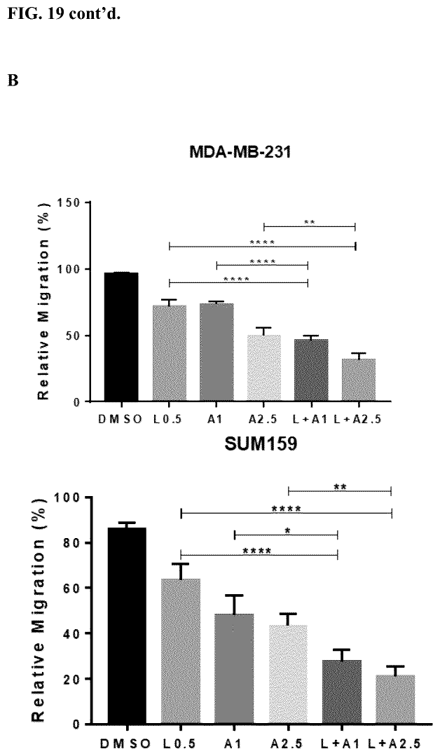

A wound healing assay showed LLL12B in combination with Abemaciclib significantly inhibited cell migration compared to single drug treatment in human triple-negative breast cancer cell lines MDA-MB-231 and SUM159 (FIG. 19, shown microscopic studies (A) and graphically via percent migration (B)). L: LLL12B; A: Abemaciclib; L0.5:0.5 μM LLL12B; A2.5:2.5 μM Abemaciclib; A1: 1 μM Abemaciclib

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application.
1. Sung, H.; Ferlay, J.; Siegel, R. L.; Laversanne, M.; Soerjomataram, I.; Jemal, A.; Bray, F. Global Cancer Statistics 2020: GLOBOCAN Estimates of Incidence and Mortality Worldwide for 36 Cancers in 185 Countries. CA Cancer J Clin 2021, 71, 209-249, doi: 10.3322/caac.21660.
2. Lam, S. W.; Jimenez, C. R.; Boven, E. Breast cancer classification by proteomic technologies: current state of knowledge. Cancer Treat Rev 2014, 40, 129-138, doi: 10.1016/j.ctrv.2013.06.006.
3. Foulkes, W. D.; Smith, I. E.; Reis-Filho, J. S. Triple-negative breast cancer. N Engl J Med 2010, 363, 1938-1948, doi: 10.1056/NEJMra1001389.
4. Dent, R.; Trudeau, M.; Pritchard, K. I.; Hanna, W. M.; Kahn, H. K.; Sawka, C. A.; Lickley, L. A.; Rawlinson, E.; Sun, P.; Narod, S. A. Triple-negative breast cancer: clinical features and patterns of recurrence. Clin Cancer Res 2007, 13, 4429-4434, doi: 10.1158/1078-0432.Ccr-06-3045.
5. McCann, K. E.; Hurvitz, S. A. Advances in the use of PARP inhibitor therapy for breast cancer. Drugs Context 2018, 7, 212540, doi: 10.7573/dic.212540.
6 Soare, G. R.; Soare, C. A. Immunotherapy for Breast Cancer: First FDA Approved Regimen. Discoveries (Craiova) 2019, 7, e91, doi: 10.15190/d.2019.4.
7. Bagegni, N. A.; Davis, A. A.; Clifton, K. K.; Ademuyiwa, F. O. Targeted Treatment for High-Risk Early-Stage Triple-Negative Breast Cancer: Spotlight on Pembrolizumab. Breast Cancer (Dove Med Press) 2022, 14, 113-123, doi: 10.2147/bctt.S293597.
8. Jhan, J. R.; Andrechek, E. R. Triple-negative breast cancer and the potential for targeted therapy. Pharmacogenomics 2017, 18, 1595-1609, doi: 10.2217/pgs-2017-0117.
9. Sporikova, Z.; Koudelakova, V.; Trojanec, R.; Hajduch, M. Genetic Markers in Triple-Negative Breast Cancer. Clin Breast Cancer 2018, 18, e841-e850, doi: 10.1016/j.clbc.2018.07.023.
10. Won, K. A.; Spruck, C. Triple-negative breast cancer therapy: Current and future perspectives (Review). Int J Oncol 2020, 57, 1245-1261, doi: 10.3892/ijo.2020.5135.
11. Qin, J. J.; Yan, L.; Zhang, J.; Zhang, W. D. STAT3 as a potential therapeutic target in triple negative breast cancer: a systematic review. J Exp Clin Cancer Res 2019, 38, 195, doi: 10.1186/s13046-019-1206-z.
12. Hsieh, F. C.; Cheng, G.; Lin, J. Evaluation of potential Stat3-regulated genes in human breast cancer. Biochem Biophys Res Commun 2005, 335, 292-299, doi: 10.1016/j.bbrc.2005.07.075.
13. Berishaj, M.; Gao, S. P.; Ahmed, S.; Leslie, K.; Al-Ahmadie, H.; Gerald, W. L.; Bornmann, W.; Bromberg, J. F. Stat3 is tyrosine-phosphorylated through the interleukin-6/glycoprotein 130/Janus kinase pathway in breast cancer. Breast Cancer Res 2007, 9, R32, doi: 10.1186/bcr1680.
14. Marotta, L. L.; Almendro, V.; Marusyk, A.; Shipitsin, M.; Schemme, J.; Walker, S. R.; Bloushtain-Qimron, N.; Kim, J. J.; Choudhury, S. A.; Maruyama, R.; et al. The JAK2/STAT3 signaling pathway is required for growth of CD44⁺CD24-stem cell-like breast cancer cells in human tumors. J Clin Invest 2011, 121, 2723-2735, doi: 10.1172/jci44745.
15. Li, L. X.; Zhou, J. X.; Calvet, J. P.; Godwin, A. K.; Jensen, R. A.; Li, X. Lysine methyltransferase SMYD2 promotes triple negative breast cancer progression. Cell Death Dis 2018, 9, 326, doi: 10.1038/s41419-018-0347-x.
16. McDaniel, J. M.; Varley, K. E.; Gertz, J.; Savic, D. S.; Roberts, B. S.; Bailey, S. K.; Shevde, L. A.; Ramaker, R. C.; Lasseigne, B. N.; Kirby, M. K.; et al. Genomic regulation of invasion by STAT3 in triple negative breast cancer. Oncotarget 2017, 8, 8226-8238, doi: 10.18632/oncotarget. 14153.
17. Moreira, M. P.; da Conceição Braga, L.; Cassali, G. D.; Silva, L. M. STAT3 as a promising chemoresistance biomarker associated with the CD44 (+/high)/CD24 (−/low)/ALDH (+) BCSCs-like subset of the triple-negative breast cancer (TNBC) cell line. Exp Cell Res 2018, 363, 283-290, doi: 10.1016/j.yexcr.2018.01.018.
18. Sasidharan Nair, V.; Toor, S. M.; Ali, B. R.; Elkord, E. Dual inhibition of STAT1 and STAT3 activation downregulates expression of PD-L1 in human breast cancer cells. Expert Opin Ther Targets 2018, 22, 547-557, doi: 10.1080/14728222.2018.1471137.
19. Demaria, M.; Giorgi, C.; Lebiedzinska, M.; Esposito, G.; D′Angeli, L.; Bartoli, A.; Gough, D. J.; Turkson, J.; Levy, D. E.; Watson, C. J.; et al. A STAT3-mediated metabolic switch is involved in tumour transformation and STAT3 addiction. Aging (Albany NY) 2010, 2, 823-842, doi: 10.18632/aging.100232.
20. Park, S. K.; Byun, W. S.; Lee, S.; Han, Y. T.; Jeong, Y. S.; Jang, K.; Chung, S. J.; Lee, J.; Suh, Y. G.; Lee, S. K. A novel small molecule STAT3 inhibitor SLSI-1216 suppresses proliferation and tumor growth of triple-negative breast cancer cells through apoptotic induction. Biochem Pharmacol 2020, 178, 114053, doi: 10.1016/j.bcp.2020.114053.
21. Sirkisoon, S. R.; Carpenter, R. L.; Rimkus, T.; Anderson, A.; Harrison, A.; Lange, A. M.; Jin, G.; Watabe, K.; Lo, H. W. Interaction between STAT3 and GLI1/tGLI1 oncogenic transcription factors promotes the aggressiveness of triple-negative breast cancers and HER2-enriched breast cancer. Oncogene 2018, 37, 2502-2514, doi: 10.1038/s41388-018-0132-4.
22. Beebe, J. D.; Liu, J. Y.; Zhang, J. T. Two decades of research in discovery of anticancer drugs targeting STAT3, how close are we? Pharmacol Ther 2018, 191, 74-91, doi: 10.1016/j.pharmthera.2018.06.006.
23. Yu, W.; Li, C.; Zhang, W.; Xia, Y.; Li, S.; Lin, J. Y.; Yu, K.; Liu, M.; Yang, L.; Luo, J.; et al. Discovery of an Orally Selective Inhibitor of Signal Transducer and Activator of Transcription 3 Using Advanced Multiple Ligand Simultaneous Docking. J Med Chem 2017, 60, 2718-2731, doi: 10.1021/acs.jmedchem.6b01489.
24. Pan, L.; Chen, X.; Fu, S.; Yu, W.; Li, C.; Wang, T.; Lo, H. W.; Lin, J. LLY17, a novel small molecule STAT3 inhibitor induces apoptosis and suppresses cell migration and tumor growth in triple-negative breast cancer. Breast Cancer Res Treat 2020, 181, 31-41, doi: 10.1007/s10549-020-05613-6.
25. Chen, X.; Pan, L.; Wei, J.; Zhang, R.; Yang, X.; Song, J.; Bai, R. Y.; Fu, S.; Pierson, C. R.; Finlay, J. L.; et al. LLL12B, a small molecule STAT3 inhibitor, induces growth arrest, apoptosis, and enhances cisplatin-mediated cytotoxicity in medulloblastoma cells. Sci Rep 2021, 11, 6517, doi: 10.1038/s41598-021-85888-x.
26. de Groot, F. M.; van Berkom, L. W.; Scheeren, H. W. Synthesis and biological evaluation of 2′-carbamate-linked and 2′-carbonate-linked prodrugs of paclitaxel: selective activation by the tumor-associated protease plasmin. J Med Chem 2000, 43, 3093-3102, doi: 10.1021/jm0009078.
27. Lin, L.; Hutzen, B.; Li, P. K.; Ball, S.; Zuo, M.; DeAngelis, S.; Foust, E.; Sobo, M.; Friedman, L.; Bhasin, D.; et al. A novel small molecule, LLL12, inhibits STAT3 phosphorylation and activities and exhibits potent growth-suppressive activity in human cancer cells. Neoplasia 2010, 12, 39-50, doi: 10.1593/neo.91196.
28. Aqel, S. I.; Yang, X.; Kraus, E. E.; Song, J.; Farinas, M. F.; Zhao, E. Y.; Pei, W.; Lovett-Racke, A. E.; Racke, M. K.; Li, C.; et al. A STAT3 inhibitor ameliorates CNS autoimmunity by restoring Teff: Treg balance. JCI Insight 2021, 6, doi: 10.1172/jci.insight.142376.
29. Matošević, A.; Bosak, A. Carbamate group as structural motif in drugs: a review of carbamate derivatives used as therapeutic agents. Arh Hig Rada Toksikol 2020, 71, 285-299, doi: 10.2478/aiht-2020-71-3466.
30 Igarashi, Y.; Yanagisawa, E.; Ohshima, T.; Takeda, S.; Aburada, M.; Miyamoto, K. Synthesis and evaluation of carbamate prodrugs of a phenolic compound. Chem Pharm Bull (Tokyo) 2007, 55, 328-333, doi: 10.1248/cpb.55.328.
31. Pan, L.; Zhang, R.; Ma, L.; Pierson, C. R.; Finlay, J. L.; Li, C.; Lin, J. STAT3 inhibitor in combination with irradiation significantly inhibits cell viability, cell migration, invasion and tumorsphere growth of human medulloblastoma cells. Cancer Biol Ther 2021, 22, 430-439, doi: 10.1080/15384047.2021.1951573.
32. Jung, K. H.; Yoo, W.; Stevenson, H. L.; Deshpande, D.; Shen, H.; Gagea, M.; Yoo, S. Y.; Wang, J.; Eckols, T. K.; Bharadwaj, U.; et al. Multifunctional Effects of a Small-Molecule STAT3 Inhibitor on NASH and Hepatocellular Carcinoma in Mice. Clin Cancer Res 2017, 23, 5537-5546, doi: 10.1158/1078-0432.Ccr-16-2253.
33. Bromberg, J.; Darnell, J. E., Jr. The role of STATs in transcriptional control and their impact on cellular function. Oncogene 2000, 19, 2468-2473, doi: 10.1038/sj.onc. 1203476.
34. Miklossy, G.; Hilliard, T. S.; Turkson, J. Therapeutic modulators of STAT signalling for human diseases. Nat Rev Drug Discov 2013, 12, 611-629, doi: 10.1038/nrd4088.
35. Butturini, E.; Carcereri de Prati, A.; Mariotto, S. Redox Regulation of STAT1 and STAT3 Signaling. Int J Mol Sci 2020, 21, doi: 10.3390/ijms21197034.
36. Avalle, L.; Pensa, S.; Regis, G.; Novelli, F.; Poli, V. STAT1 and STAT3 in tumorigenesis: A matter of balance. Jakstat 2012, 1, 65-72, doi: 10.4161/jkst.20045.
37. Guanizo, A. C.; Fernando, C. D.; Garama, D. J.; Gough, D. J. STAT3: a multifaceted oncoprotein. Growth Factors 2018, 36, 1-14, doi: 10.1080/08977194.2018.1473393.
38. Lieblein, J. C.; Ball, S.; Hutzen, B.; Sasser, A. K.; Lin, H. J.; Huang, T. H.; Hall, B. M.; Lin, J. STAT3 can be activated through paracrine signaling in breast epithelial cells. BMC Cancer 2008, 8, 302, doi: 10.1186/1471-2407-8-302.
39. Wake, M. S.; Watson, C. J. STAT3 the oncogene-still eluding therapy? Febs j 2015, 282, 2600-2611, doi: 10.1111/febs. 13285.
40. Furtek, S. L.; Backos, D. S.; Matheson, C. J.; Reigan, P. Strategies and Approaches of Targeting STAT3 for Cancer Treatment. ACS Chem Biol 2016, 11, 308-318, doi: 10.1021/acschembio.5b00945.
41. Wong, A. L. A.; Hirpara, J. L.; Pervaiz, S.; Eu, J. Q.; Sethi, G.; Goh, B. C. Do STAT3 inhibitors have potential in the future for cancer therapy? Expert Opin Investig Drugs 2017, 26, 883-887, doi: 10.1080/13543784.2017.1351941.
42. Schust, J.; Sperl, B.; Hollis, A.; Mayer, T. U.; Berg, T. Stattic: a small-molecule inhibitor of STAT3 activation and dimerization. Chem Biol 2006, 13, 1235-1242, doi: 10.1016/j.chembiol.2006.09.018.
43. Siddiquee, K.; Zhang, S.; Guida, W. C.; Blaskovich, M. A.; Greedy, B.; Lawrence, H. R.; Yip, M. L.; Jove, R.; McLaughlin, M. M.; Lawrence, N. J.; et al. Selective chemical probe inhibitor of Stat3, identified through structure-based virtual screening, induces antitumor activity. Proc Natl Acad Sci USA 2007, 104, 7391-7396, doi: 10.1073/pnas.0609757104.
44. Chen, Y.; Ji, M.; Zhang, S.; Xue, N.; Xu, H.; Lin, S.; Chen, X. Bt354 as a new STAT3 signaling pathway inhibitor against triple negative breast cancer. J Drug Target 2018, 26, 920-930, doi: 10.1080/1061186x.2018.1452244.
45. Zhang, R.; Yang, X.; Roque, D. M.; Li, C.; Lin, J. A novel small molecule LLL12B inhibits STAT3 signaling and sensitizes ovarian cancer cell to paclitaxel and cisplatin. PLOS One 2021, 16, e0240145, doi: 10.1371/journal.pone.0240145.

1-41. (canceled)

42. A method of treating cancer in a subject, comprising administering a therapeutically-effective amount of LLL12B to a subject in need thereof, thereby treating cancer in a subject.

43. The method of claim 42, wherein cells of the cancer persistently or constitutively expresses STAT3 (signal transducer and activator of transcription 3).

44. The method of claim 42, wherein the cancer is breast cancer or pancreatic cancer.

45. The method of claim 44, wherein the breast cancer is triple-negative breast cancer (TNBC).

46. The method of claim 42, wherein the therapeutically-effective amount of LLL12B is sufficient to achieve one or more of (i) induce apoptosis in cells of the cancer, (ii) inhibit or block growth of cells of the cancer, (iii) inhibit or block migration of cells of the cancer, (iv) inhibit or block STAT3 activity in cells of the cancer, (v) inhibit or block STAT3 signaling in cells of the cancer, (vi) inhibit or block STAT3 phosphorylation in cells of the cancer, and (vii) inhibit or block STAT3 phosphorylation-induction activation in cells of the cancer.

47. The method of claim 42, wherein the therapeutically-effective amount of LLL12B is an amount ranging from about 1 mg/kg to 20 mg/kg, body weight.

48. A method of treating cancer in a subject, comprising administering a therapeutically-effective amount of LLL12B and one or more additional therapeutic agents to a subject in need thereof, thereby treating cancer in a subject.

49. The method of claim 48, wherein the one or more additional therapeutic agents are selected from PARP inhibitors and CDK inhibitors.

50. The method of claim 49, wherein the PARP inhibitors are selected from the group consisting of talazoparib, olaparib, rucaparib, veliparib and niraparib.

51. The method of claim 49, wherein the CDK inhibitors are selected from the group consisting of abemaciclib, palbociclib, ribociclib, Alvocidib, Dinaciclib, P276-00, AT7519 and Roniciclib.

52. The method of claim 48, wherein cells of the cancer persistently or constitutively expresses STAT3.

53. The method of claim 48, wherein cells of the cancer is breast cancer or pancreatic cancer.

54. The method of claim 53, wherein the breast cancer is TNBC.

55. The method of claim 48, wherein the therapeutically-effective amount of LLL12B is sufficient to achieve one or more of (i) induce apoptosis in cells of the cancer, (ii) inhibit or block growth of cells of the cancer, (iii) inhibit or block migration of cells of the cancer, (iv) inhibit or block STAT3 activity in cells of the cancer, (v) inhibit or block STAT3 signaling in cells of the cancer, (vi) inhibit or block STAT3 phosphorylation in cells of the cancer, and (vii) inhibit or block STAT3 phosphorylation-induction activation in cells of the cancer.

56. The method of claim 48, wherein the therapeutically-effective amount of LLL12B is an amount ranging from about 1 mg/kg to 20 mg/kg, body weight.

57. The method of claim 49, wherein the amount of the PARP inhibitor is an amount ranging from about 0.05 to about 15 mg/kg, body weight.

58. The method of claim 49, wherein the amount of the CDK inhibitor is an amount ranging from about 10 to about 250 mg/kg, body weight.

59. The method of claim 48, wherein the combination of LLL12B and the additional therapeutic agents has a synergistic therapeutic effect on the cancer.

60. A method of inhibiting STAT3 activity in vivo, comprising administering to a subject having cells expressing STAT3 an amount of LLL12B effective to inhibit STAT3 activity, thereby inhibiting STAT3 activity in vivo.

61. The method of claim 60, wherein the cells expressing STAT3 are cancer cells persistently or constitutively expressing STAT3.

62. The method of claim 60, wherein the cells expressing STAT3 are breast cancer cells or pancreatic cancer cells.

63. The method of claim 62, wherein the breast cancer is TNBC.

64. The method of claim 60, wherein the amount of LLL12B effective to inhibit STAT3 activity is an amount ranging from about 1 mg/kg to 20 mg/kg, body weight.

65. The method of claim 60, further comprising administering to the subject an effective amount of one or more additional therapeutic agents.

66. The method of claim 65, wherein the one or more additional therapeutic agents are selected from PARP inhibitors and CDK inhibitors.

67. The method of claim 66, wherein the PARP inhibitors are selected from the group consisting of talazoparib, olaparib, rucaparib, veliparib and niraparib.
